(19) United States
(12) Patent Application Publication
(10) Pub. No.: US 2009/0311695 A1
(43) Pub. Date: Dec. 17, 2009

(54) Method

(76) Inventors: Talat Nasim, London (GB); Richard Charles Trembath, London (GB)

Correspondence Address: STEPTOE & JOHNSON LLP, 1330 CONNECTICUT AVENUE, N.W., WASHINGTON, DC, 20036, US

(21) Appl. No.: 12/403,092

(22) Filed: Mar. 12, 2009

Related U.S. Application Data
(60) Provisional application No. 61/035,855, filed on Mar. 12, 2008.

(51) Int. Cl. C12Q 1/68; C07H 21/00; C12N 15/74; C12N 5/10

(52) U.S. Cl. 435/6; 536/23.1; 435/320.1; 435/325

(57) ABSTRACT

In one aspect, there is described a method for determining the effect of a genetic variation or mutation on the integrity of an RNA transcript comprising the steps of: (a) providing a nucleic acid construct comprising two different reporters separated by an in frame splicing unit, wherein said splicing unit comprises at least two exons separated by at least one intron and wherein at least one exon (eg. the exon downstream of said intron) comprises the genetic variation; (b) transfecting said construct into a cell and/or generating a stable cell line; (c) culturing said cell; and (d) determining the effect of said genetic variation on the integrity of the RNA transcript, wherein a difference in reporter activity in comparison to a cell comprising the nucleic acid construct without the genetic variation is indicative that said genetic variation affects the integrity of the RNA transcript. Assays—such as high throughput assays—are also described for identifying agents (nucleic acids, peptides and small molecules) that modulate the integrity of the RNA transcript and/or are involved in modulating RNA metabolism.

### CLAIM OF PRIORITY

This application claims the benefit of prior U.S. Provisional Application No. 61/035,855, filed on Mar. 12, 2008, which is incorporated by reference in its entirety.

### FIELD OF INVENTION

The present invention relates to a method(s) for determining the effect of a genetic variation on the integrity of an RNA transcript and/or on RNA metabolism. Assays—such as high throughput assays—are also described for identifying agents that modulate the integrity of the RNA transcript and/or are involved in modulating RNA metabolism.

### INTRODUCTION

An outstanding challenge in the assessment of the increasing availability of human genome sequence is the demanding requirement for functional annotation of sequence variation particularly in regard of efforts to establish a potential pathogenic role.

The spectrum of mutations identified in human diseases includes single nucleotide substitutions, deletions, consensus splice site variations, small insertion or deletion, partial or complete exon deletion or duplication. Each of them can alter transcript integrity (1). Approximately 33% of mutant alleles that contribute to human disease harbour premature termination codons (PTCs) are predicted to preclude the synthesis of a full-length protein (2). In contrast, the majority of PTC harbouring transcripts produce significantly less proteins as the transcript abundance is reduced by a process called nonsense mediated decay (NMD) (3). Some nonsense mutations cause exon skipping, presumably during splicing and hence are termed nonsense-associated splicing (NAS) defects (4). The underlying mechanisms of NAS are not clearly understood, but it is thought that nuclear scanning recognizes PTCs in the nucleus and alters splicing to skip the nonsense-containing exon. Increasing evidence suggests that nonsense mutation(s) may disrupt exonic and intronic splicing enhancer (ESE and ISE) or create exonic and intronic splicing suppressors (ESS and ISS) (5). These events may also lead to aberrant splicing (6), and generate proteins with altered functions. The characterization of molecular consequences of genome sequence variation found in human diseases depends at least in part upon understanding whether (nonsense) mutations activate aberrant splicing or the (nonsense) transcripts are decayed through the NMD pathway.

Methods for validating transcript integrity at the levels of splicing and NMD include direct analysis of transcripts by means of reverse transcriptase PCR analysis, which do not lend themselves for high-throughput screening. Single reporters linked to enzymatic or fluorescence activities have been developed but they are susceptible to numerous intrinsic variables in the levels of transfection, transcription and translation (7), (8), (9), (10). To overcome these difficulties we previously developed a double reporter splicing system which minimizes the variables confounding single reporter functions (11), (12) for determining splicing efficiency.

There is currently lacking a rapid definitive test to determine if a mutation—such as a genome sequence variation—affects transcript integrity or RNA metabolism at the level of splicing and NMD.

There is also increasing interest in the identification of small molecules that activate translation readthrough of nonsense associated human diseases. For example, acetylamino benzoic acid, oxadiazole benzoic acid and aminoglycosides have been found to promote translation readthrough in cystic fibrosis and muscular dystrophy patients, mammalian cell lines and mouse models. High throughput assays to rapidly screen for such molecules are required.

The present invention seeks to overcome the problems of the prior art.

### SUMMARY OF THE INVENTION

There is described herein assays based on the use of reporter systems eg. indirect reporter systems, which utilize a dual-fluorescence assay whereby the effect of sequence variation can be visualized in, for example, single live mammalian cells. This provides an instant insight into whether the variation leads to such processes as splicing or aberrant splicing or triggers the NMD pathway. Using these assays it has been found that contrary to computer simulation predictions, nonsense, insertion and deletion mutations do not stimulate aberrant splicing but trigger the NMD process. The assay systems described herein may be useful for the rapid investigation of the consequences of germ line mutations in disease and will aid the identification of factors that can restore a normal expression pattern specifically by targeting splicing, translation and NMD machineries. In particular, these assays have potential utility for screening agents—such as cDNA, chemical and/or peptide libraries—to identify agents that promote or inhibit processes—such as splicing, translation read-through and NMD.

Advantageously, the assay systems described herein offer a new and improved approach for the functional characterization of genome sequence variations—in disease. The methods described herein can also be used to determine if a sequence variation activates, for example, either aberrant splicing or triggers the NMD pathway in living cells. Accordingly, this overcomes the need to use individual techniques to determine, for example, splicing or NMD processes.

### SUMMARY ASPECTS OF THE PRESENT INVENTION

There is provided a method for determining the effect of a genetic variation on the integrity of an RNA transcript comprising the use of a nucleic acid construct comprising two different reporters separated by an in frame splicing unit, wherein said splicing unit comprises at least two exons separated by at least one intron and wherein at least one exon comprises a genetic variation, wherein a difference in reporter activity in a cell comprising the nucleic acid construct in comparison to a cell comprising a nucleic acid construct without the genetic variation is indicative that said genetic variation affects the integrity of the RNA transcript.

There is also provided a method for identifying at least one agent that modulates splicing comprising the use of a nucleic acid construct comprising at least two different reporters separated by an in frame splicing unit, wherein said splicing unit comprises at least two exons separated by at least one intron and wherein the at least one intron does not comprise one or more stop codon(s), wherein a difference in reporter activity in a cell comprising the nucleic acid construct that has been exposed to the agent in comparison to a cell that has not been exposed to the agent is indicative that said agent modulates splicing.

There is also provided a method for identifying at least one agent that modulates translation comprising the use of a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon, wherein a difference in reporter activity in a cell comprising the nucleic acid construct that is exposed to the agent in comparison to a cell that has not been exposed to the agent is indicative that said agent modulates translation.

There is also provided a method for identifying at least one agent that modulates translation and/or nonsense mediated decay comprising the use of a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon, wherein a difference in reporter activity in a cell comprising the nucleic acid construct that is exposed to the agent in comparison to a cell that has not been exposed to the agent is indicative that said agent modulates translation and nonsense mediated decay.

There is also provided a method for identifying at least one agent that modulates exon incorporation or exon skipping comprising the use of a nucleic acid construct comprising at least two different reporters separated by an in frame splicing unit, wherein said splicing unit comprises at least three exons separated by at least two introns, wherein a difference in reporter activity in a cell comprising the nucleic acid construct that is exposed to the agent in comparison to a cell that has not been exposed to the at least one agent is indicative that said agent modulates exon skipping or exon incorporation.

There is also provided a method for identifying at least one agent that modulates exon incorporation or exon skipping or nonsense mediated decay comprising the use of a nucleic acid construct comprising two different reporters separated by an in frame splicing unit, wherein said splicing unit comprises three exons separated by two introns, wherein at least one exon (eg. the exon separated by the two introns) comprises a premature termination codon, wherein a difference in reporter activity in a cell comprising the nucleic acid construct that is exposed to the agent in comparison to a cell that has not been exposed to the agent is indicative that said agent modulates exon incorporation or exon skipping or nonsense mediated decay.

There is also provided a method for identifying at least one agent that modulates nonsense mediated decay comprising the use of a nucleic acid construct comprising two different reporters separated by an in frame splicing unit, wherein said splicing unit comprises three exons separated by two introns, wherein at least one exon (eg. the exon separated by the two introns) comprises a premature termination codon, wherein a difference in reporter activity in a cell comprising the nucleic acid construct that has been exposed to the agent in comparison to a cell that has not been exposed to the at least one agent is indicative that said agent modulates nonsense mediated decay.

There is also provided an agent obtained or obtainable by the methods described herein.

There is also provided a nucleic acid construct comprising two different reporters separated by an in frame splicing unit, wherein said splicing unit comprises at least two exons separated by at least one intron, with the proviso that said intron does not comprise one or more translation stop signal(s).

There is also provided a nucleic acid construct comprising two different reporters separated by an in frame splicing unit, wherein said splicing unit comprises at least two exons separated by at least one intron and wherein at least one exon (eg. the exon downstream of said intron) comprises a mutation.

There is also provided a nucleic acid construct comprising two different reporters separated by an in frame splicing unit, wherein said splicing unit comprises three exons separated by two introns, wherein at least one exon (eg. the exon separated by the two introns) comprises at least one mutation that causes nonsense mediated decay.

There is also provided a vector comprising the nucleic acid construct described herein.

There is also provided a host cell comprising the nucleic acid construct or the vector described herein.

There is also provided a method for determining the effect of at least one mutation on the integrity of an RNA transcript comprising the use of the nucleic acid construct or the vector or the cell described herein.

There is also provided method for identifying an agent that modulates the integrity of an RNA transcript comprising the use of the nucleic acid construct, the vector or the cell as described herein.

There is also provided the use of the nucleic acid construct, the vector or the cell described herein for determining the effect of at least one mutation on the integrity of an RNA transcript.

There is also provided the use of the nucleic acid construct, the vector or the cell described herein for identifying an agent that modulates the integrity of an RNA transcript.

There is also provided mutation c.2292insA in exon 12 of BMPR2.

There is also provided mutation c.2386delG in exon 12 of BMPR2.

There is also provided mutation c.2695C>T in exon 12 of BMPR2.

There is also provided mutation c.2386delG in exon 12 of BMPR2.

There is also provided mutation c.2620G>T in exon 12 of BMPR2.

There is also provided a method, an agent, a nucleic acid construct, a vector, a host cell, a use or a mutation as described herein with reference to the accompanying description and drawings.

### SUMMARY EMBODIMENTS OF THE PRESENT INVENTION

Suitably, the method according to the first aspect comprises the steps of: (a) providing a nucleic acid construct comprising two different reporters separated by an in frame splicing unit, wherein said splicing unit comprises at least two exons separated by at least one intron and wherein at least one exon (eg. the exon downstream of said intron) comprises the genetic variation; (b) transfecting said construct into a cell; (c) culturing said cell; and (d) determining the effect of said genetic variation on the integrity of the RNA transcript, wherein a difference in reporter activity in comparison to a cell comprising the nucleic acid construct without the genetic variation is indicative that said genetic variation affects the integrity of the RNA transcript.

Suitably, expression of both reporters is indicative that said genetic variation does not terminate translation and is not a premature termination codon.

Suitably, expression of the single reporter upstream of the splicing unit is indicative that said genetic variation terminates translation and is a premature termination codon.

Suitably, step (a) comprises providing a nucleic acid construct comprising two different reporters separated by an in frame splicing unit, wherein said splicing unit comprises three exons separated by two introns, wherein at least one exon (eg. the exon separated by the two introns) comprises the genetic variation.

Suitably, expression of both reporters is indicative that said genetic variation results in exon incorporation or exon skipping and is not a premature termination codon.

Suitably, exon incorporation and/or exon skipping are further distinguished using RT-PCR.

Suitably, expression of the single reporter upstream of the splicing unit in combination with the exons upstream of said genetic variation is indicative that said genetic variation is a premature termination codon and that the RNA transcript is subject to nonsense mediated decay.

Suitably, wherein the genetic variation is a mutation.

Suitably, the mutation is a nonsense mutation, an insertion mutation, a deletion mutation or a substitution mutation.

Suitably, the reporter(s) is/are fluorescent reporters.

Suitably, at least one of the reporters is DsRedExpress.

Suitably, at least one of the reporters is GFP.

Suitably, the method according to the second aspect comprises the steps of: (a) providing a nucleic acid construct comprising at least two different reporters separated by an in frame splicing unit, wherein said splicing unit comprises at least two exons separated by at least one intron and wherein the at least one intron does not comprise a stop codon; (b) transfecting said construct into a cell; (c) culturing said cell; (d) contacting said cell with at the least one agent; and (e) determining the effect of said agent on the splicing of RNA, wherein a difference in reporter activity in comparison to a cell that has not been exposed to the at least one agent is indicative that said agent modulates splicing.

Suitably, expression of both reporters in the cell exposed to the agent and the expression of a single reporter in the cell not exposed to the agent is indicative that said agent modulates (eg. promotes) normal splicing.

Suitably, an increase in the ratio of the expression of both reporters in the cell exposed to the agent in comparison to the cell not exposed to the agent is indicative that said agent promotes normal splicing.

Suitably, expression of a single reporter in the cell exposed to the agent and the expression of both reporters in the cell not exposed to the agent is indicative that said agent promotes splicing inhibition.

Suitably, an increase in the expression of the single reporter in the cell exposed to the agent in comparison to the cell not exposed to the agent is indicative that said agent promotes splicing inhibition.

Suitably, the method according to the third aspect comprises the steps of: (a) providing a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon; (b) transfecting said construct into a cell and/or establishing a stable cell line; (c) culturing said cell; (d) contacting said cell with the at least one agent; and (e) determining the effect of said agent on the splicing of RNA; wherein a difference in reporter activity in comparison to a cell that has not been exposed to the at least one agent is indicative that said agent modulates translation.

Suitably, expression of both reporters in the cell exposed to the agent and the expression of a single reporter in the cell not exposed to the agent is indicative that said agent promotes translational read-through.

Suitably, an increase in the ratio of the expression of both reporters in the cell exposed to the agent in comparison to the cell not exposed to the agent is indicative that said agent promotes translation read-through.

Suitably, expression of a single reporter in the cell exposed to the agent and the expression of both reporters in the cell not exposed to the agent is indicative that said agent promotes translation termination.

Suitably, an increase in the expression of the single reporter in the cell exposed to the agent in comparison to the cell not exposed to the agent is indicative that said agent promotes translation termination.

Suitably, expression of both reporters in the cell exposed to the agent and the expression of a single reporter in the cell not exposed to the agent is indicative that said agent promotes translational read-through and inhibits nonsense mediated decay.

Suitably, an increase in the expression (ratio) of both reporters in the cell exposed to the agent in comparison to the cell not exposed to the agent is indicative that said agent promotes promotes translational read-through and inhibits nonsense mediated decay.

Suitably, expression of a single reporter in the cell exposed to the agent and the expression of both reporters in the cell not exposed to the agent is indicative that said agent promotes translation termination.

Suitably, an increase in the expression of the single reporter in the cell exposed to the agent in comparison to the cell not exposed to the agent is indicative that said agent promotes translation termination.

Suitably, the method according to the sixth aspect comprises the steps of: (a) providing a nucleic acid construct comprising at least two different reporters separated by an in frame splicing unit, wherein said splicing unit comprises at least three exons separated by at least two introns; (b) transfecting said construct into a cell and/or establishing a stable cell line; (c) culturing said cell; (d) contacting said cell with at least one agent; and (e) determining the effect of said mutation on exon skipping; wherein a difference in reporter activity in comparison to a cell that has not been exposed to the at least one agent is indicative that said agent modulates exon skipping or exon incorporation.

Suitably, wherein expression of both reporters in the cell exposed to the agent and the expression of a single reporter in the cell not exposed to the agent is indicative that said agent promotes exon incorporation.

Suitably, an increase in the expression of both reporters in the cell exposed to the agent in comparison to the cell not exposed to the agent is indicative that said agent promotes exon incorporation.

Suitably, expression of a single reporter in the cell exposed to the agent and the expression of both reporters in the cell not exposed to the agent is indicative that said agent promotes exon skipping and/or alternative splice site selection.

Suitably, an increase in the expression of the single reporter in the cell exposed to the agent in comparison to the cell not exposed to the agent is indicative that said agent promotes exon skipping and/or alternative splice site selection.

Suitably, the method according to the seventh aspect comprises the steps of: (a) providing a nucleic acid construct comprising two different reporters separated by an in frame splicing unit, wherein said splicing unit comprises three exons separated by two introns, wherein at least one exon (eg. the exon separated by the two introns) comprises a premature termination codon; (b) transfecting said construct into a cell and/or establishing a stable cell line; (c) culturing said cell; (d) contacting said cell with at least one agent; and (e) determining the effect of said mutation; wherein a difference in reporter activity in comparison to a cell that has not been exposed to the at least one agent is indicative that said agent modulates exon incorporation or exon skipping or nonsense mediated decay

Suitably, expression of both reporters in the cell exposed to the agent and the expression of a single reporter in the cell not exposed to the agent is indicative that said agent promotes exon incorporation or exon skipping.

Suitably, an increase in the expression of both reporters in the cell exposed to the agent in comparison to the cell not exposed to the agent is indicative that said agent promotes exon incorporation or exon skipping.

Suitably, exon incorporation or exon skipping is verified by analysing the RNA transcript.

Suitably, the RNA transcript is analyzed using RT-PCR.

Suitably, expression of a single reporter in the cell exposed to the agent and the expression of both reporters in the cell not exposed to the agent is indicative that said agent promotes nonsense mediated decay.

Suitably, an increase in the expression of the single reporter in the cell exposed to the agent in comparison to the cell not exposed to the agent is indicative that said agent promotes nonsense mediated decay.

Suitably, the method according to the eighth aspect comprises the steps of: (a) providing a nucleic acid construct comprising two different reporters separated by an in frame splicing unit, wherein said splicing unit comprises three exons separated by two introns, wherein at least one exon (eg. the exon separated by the two introns) comprises a premature termination codon; (b) transfecting said construct into a cell and/or establishing a stable cell line; (c) culturing said cell; (d) contacting said cell with at least one agent; and (e) determining the effect of said agent on nonsense mediated decay; wherein a difference in reporter activity in comparison to a cell that has not been exposed to the at least one agent is indicative that said agent modulates nonsense mediated decay.

Suitably, a low level of the reporter upstream of the splicing unit in the cell exposed to the at least one agent in comparison to the cell that has not been exposed to the at least one agent is indicative that said agent promotes nonsense mediated decay.

Suitably, a high level of the reporter upstream of the splicing unit in the cell exposed to the at least one agent in comparison to the cell that has not been exposed to the at least one agent is indicative that said agent inhibits nonsense mediated decay.

BRIEF DESCRIPTION OF THE FIGURES

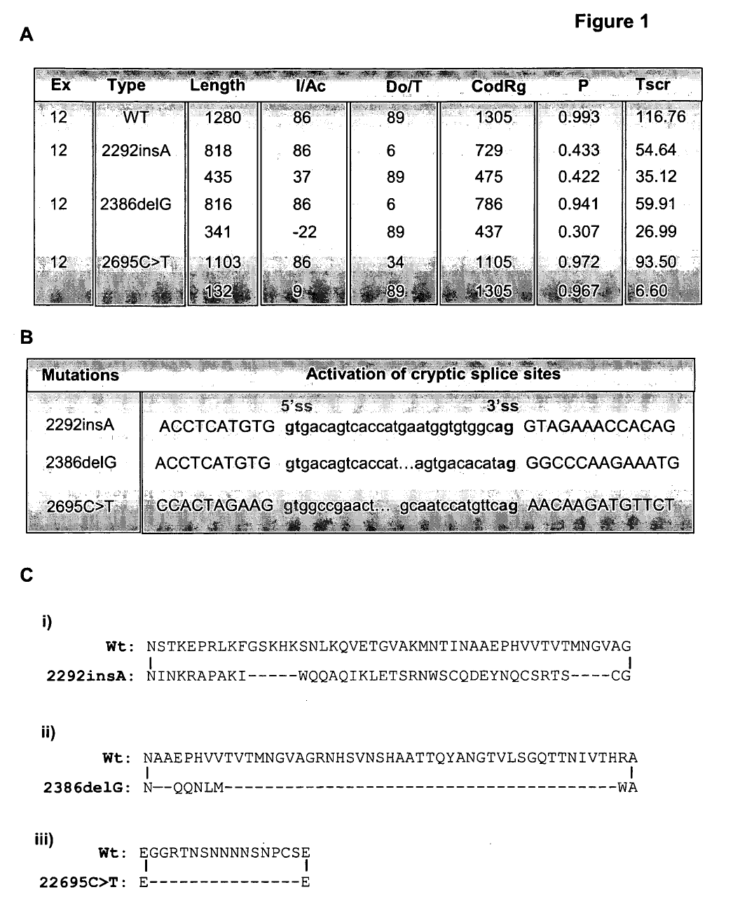

FIG. 1

Effect of mutation on transcript integrity as predicted by Genscan. The program correctly predicts the 5′ and 3′ splice sites of exon 12 of the BMPR2 gene. It also predicts that the insertion (c.2292insA), deletion (c.2386delG) and nonsense mutations (c.2695C>T) each may alter the strength of 5′ (Do/T) and 3′ (I/Ac) splice sites, exon score probability (P), T-score (exon score) and the length of the exon (FIG. 1A) and as a result activate cryptic splice sites (FIG. 1B). Exon and intron sequences are depicted in upper and lower case, respectively. 5′ss, 5′ splice site; 3′ss, 3′ splice site. As a consequence this would lead to the skipping of the nonsense codon and hence maintain a reading frame (FIG. 1C). The amino acid sequence of the mutant protein is aligned with the corresponding fragment of wild type (WT) BMPR-II protein.

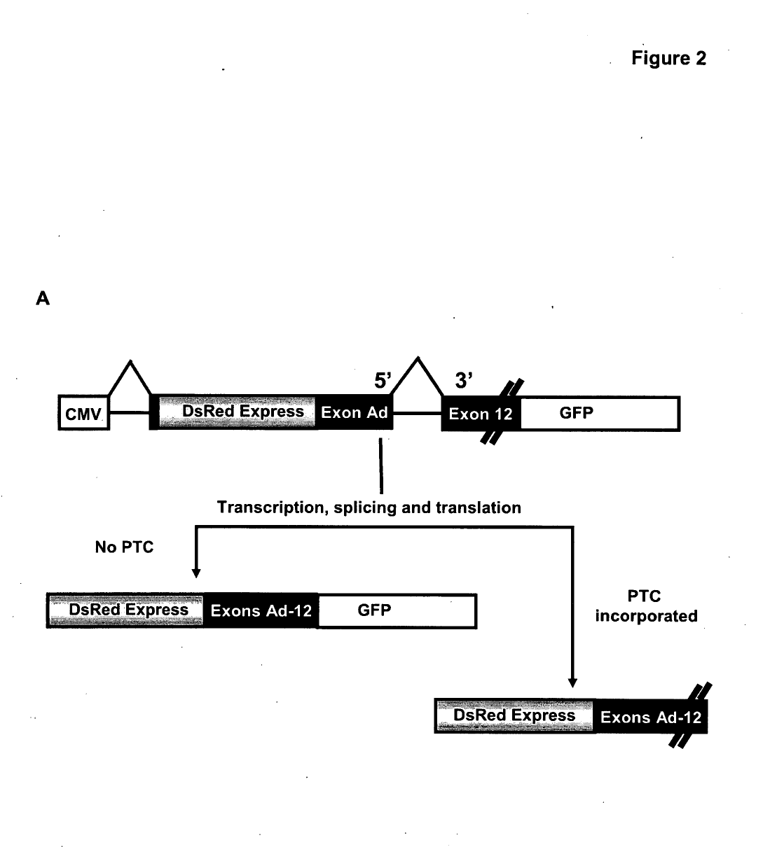

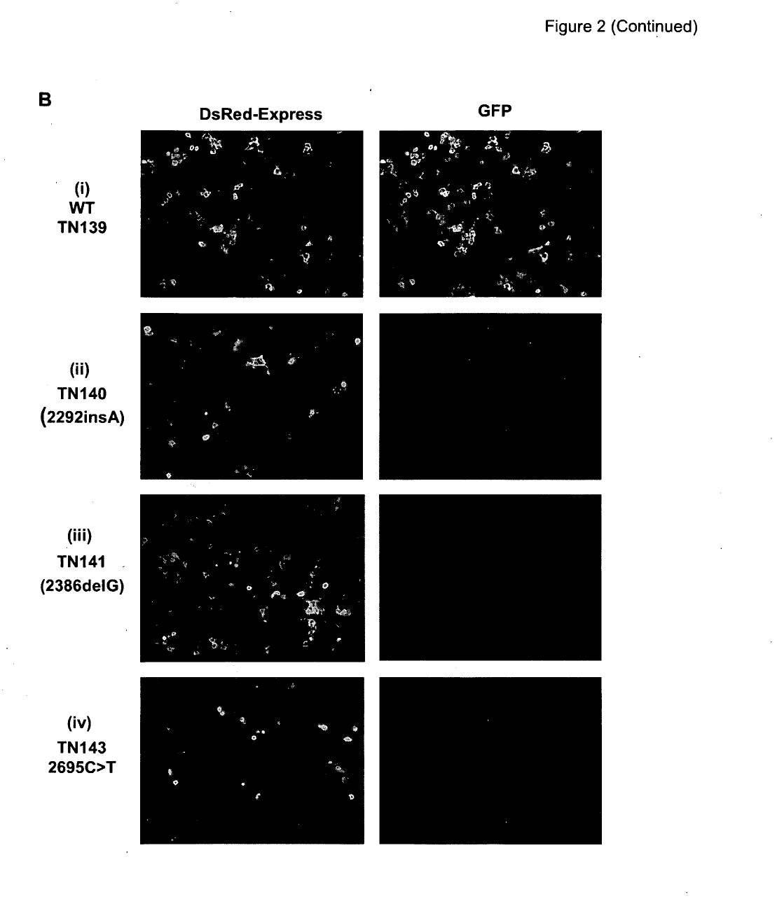

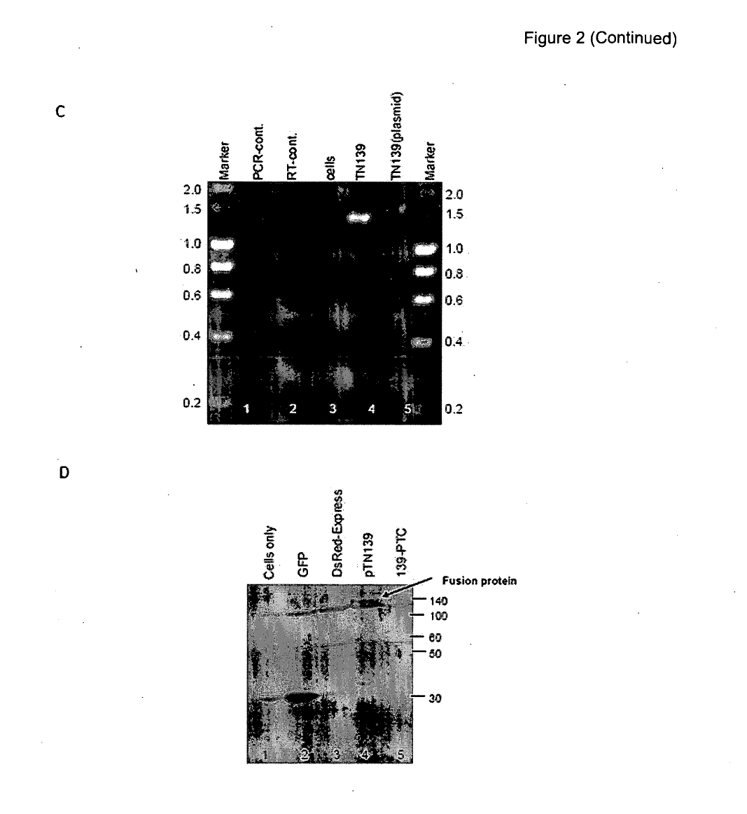

FIG. 2

Effect of sequence variation on BMPR2 transcript integrity determined by the dual-light assay. (A) Diagram of the single-cell based dual-fluorescence assay system. The Exon 12 of BMPR2 was introduced in a manner such that efficient splicing produced a DsRed-GFP fusion protein. Introduction of premature termination codon (PTC) into the exon 12 could lead splicing in either of two ways. In the event of PTC-associated skipping, as mentioned in FIG. 1, a dual-fluorescence protein was produced. In the event that recognized PTC as translational stop codon, would lead to the production of the DsRed-Express protein. (B) HEK 293 cells transfected with the wild type construct (pTN139) generated both DsRed-Express and GFP fluorescence proteins (FIG. 2Bi) whereas insertion (c.2292insA; pTN140; FIG. 2Bii)), deletion (c.2386delG; pTN141; FIG. 2Biii) and nonsense mutation (c.2695C>T; pTN143; FIG. 2Biv) only produced Ds-Red Express protein. (C) The total RNA isolated from cells transfected with pTN139 was digested with DNAse I and Hind III to remove residual plasmid DNA contaminant and analyzed by RT-PCR demonstrated that the band derived from spliced RNA. RT Cont. and PCR cont. are reactions where no reverse transcriptase enzyme and cDNA, respectively was added to the reaction. (D) Western blot analysis of dual-fluorescence protein. Cell lysates from HEK 293 cells following transfection were run on a 12% SDS gel, blotted onto nitrocellulose membrane and hybridized with anti-GFP antibody. The GFP antibody detects a band in cells transfected with GFP plasmid (lane 2). Lane 1 and 3 are lysates from mock transfected control and DsRed-Express transfected cells, respectively. The dual fluorescence reporter generates a fusion protein of approximately 140 KDa (lane 3), which is absent in construct containing a nonsense codon (lane 4). Bands migrating approximately at 125, 55, 30 are non-specific as they appeared on mock-transfected control cells.

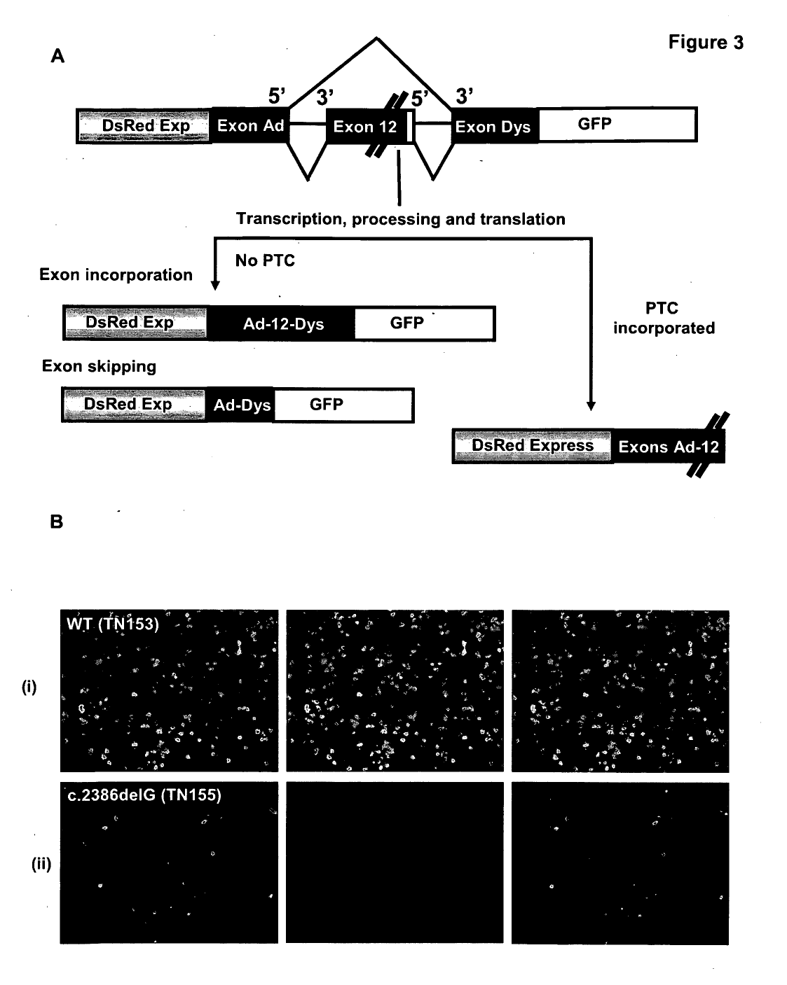

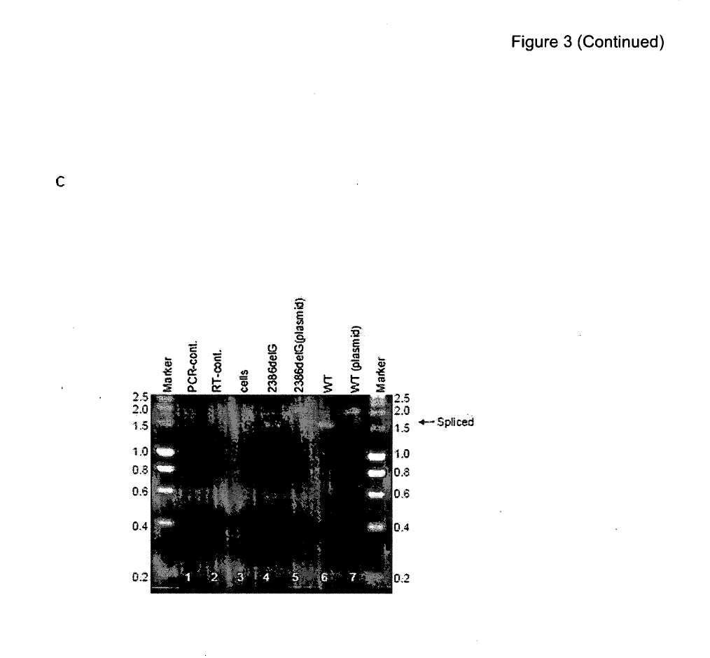

FIG. 3

The double intron based dual-fluorescence assay. The assay was constructed in such a way that the central exon was cloned in frame with the upstream and downstream exons (A) such that it maintains the reading frame. Upon transcription from the CMV promoter, PTC and non-PTC bearing the mRNAs could be processed in either of two ways. Introduction of PTC into the exon 12 would lead to the production of the upstream reporter. Non-PTC bearing mRNAs derived from exon incorporation and skipping events would generate a fusion protein with dual-fluorescence activities. (B) Cells expressing wild type construct (BMPR2 wild type; pTN153; FIG. 3Bi) generated dual fluorescence. In contrast cells expressing mutant construct (c.2386delG; pTN155; electrophoresis as described in 2C.

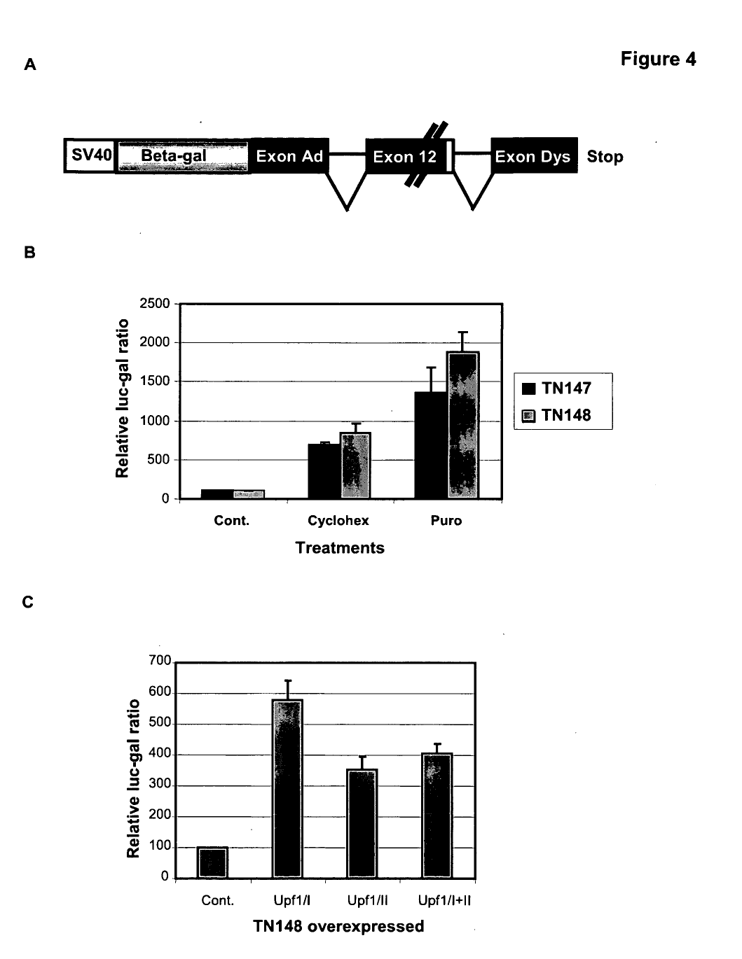

FIG. 4

(A) The test system for determining nonsense mediated RNA decay in mammalian cells based on the reporter gene encoding β-galactosidase, which was fused in-frame with the PTC (c.2386delG, c.2292insA) bearing recombinant double intron splicing unit (FIG. 4A). Plasmids bearing c.2292insA (pTN147) and c.2386delG (pTN148) mutations were transfected into HE 293 cells and grown in the presence or absence of NMD inhibitors including puromycin and cycloheximide. The treatments increased the gal-luc ratio of the reporter constructs between 5 and 20 fold (FIG. 4B) compared with untreated control. Compared with cells transfected with the reporter alone, the gal-luc ratio increased between 3 and 6 fold when hUpf1 RNA was knock down by RNAi (FIG. 4C).

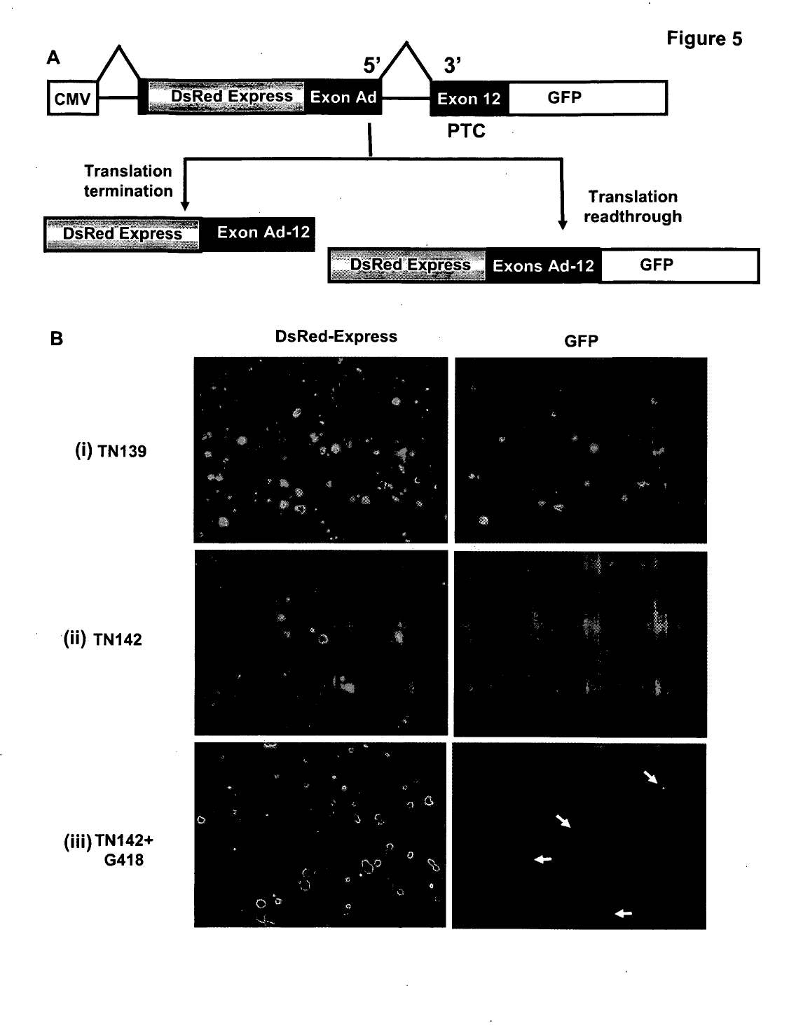

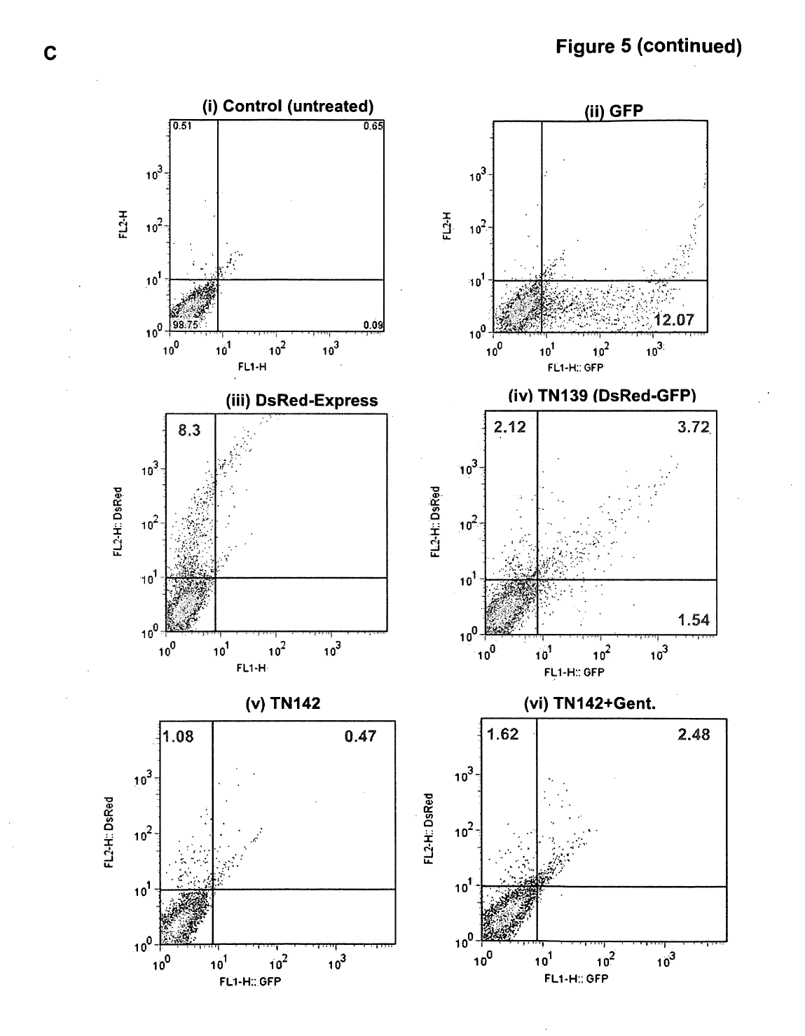

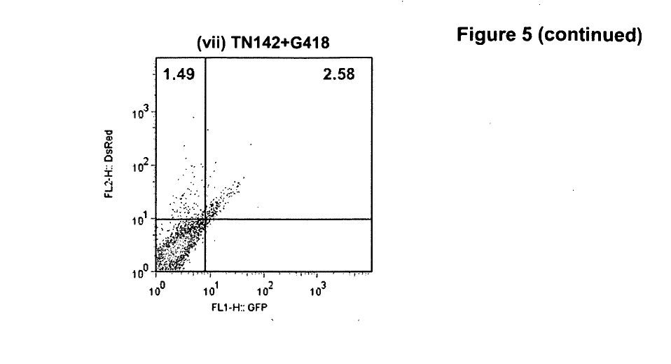

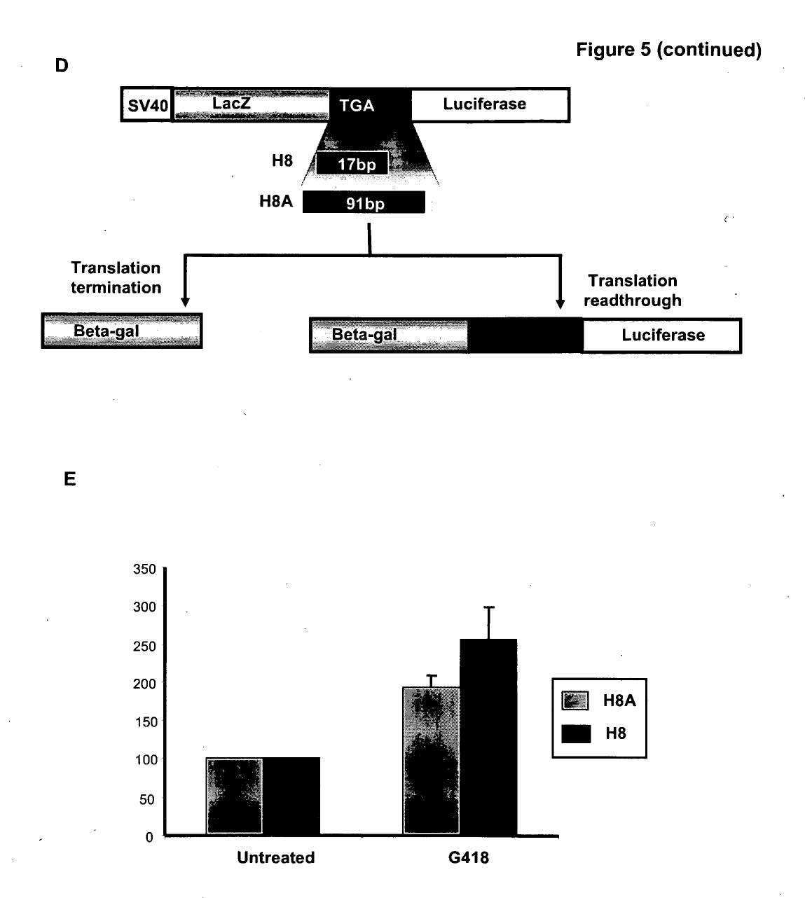

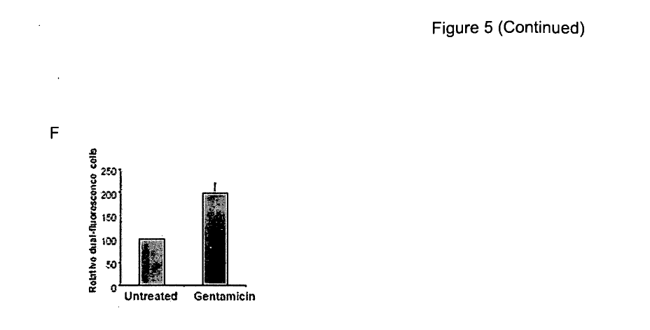

FIG. 5

(A) An outline of the single cell-based translation readthrough assay. The principle of the reporter system is similar to that mentioned in FIG. 2 with the exception that it contains a translation termination signal in the exon 12. In the event of translation termination at the internal PTC, a DsRed-Express protein will be generated whilst readthrough will produce a fusion dual-fluorescence protein (DsRed Express and GFP). Thus the ratio of two fluorescence intensities determines the efficiency of readthrough. 293 cells transfected with wild type construct (pTN139) generated dual-fluorescence (FIG. 5Bi), whilst construct containing internal PTC produced single fluorescence (FIG. 5Bii). (C) Cell expressing fluorescence proteins were analyzed with FACS. Cell transfected with pTN139 generated the highest population of dual-fluorescence cells (FIG. 5Civ), whilst pTN142 produced no or few dual-fluorescence cells. The population of TN142 derived dual-fluorescence cells was markedly increased following gentamicin (FIGS. 5Cvi and F) and G418 sulphate (FIG. 5Cvii) treatments. GFP (FIG. 5Cii) and DsRed-Express (FIG. 5Ciii) proteins expressing cells were used as fluorescence positive control, whilst untransfected cells (FIG. 5Ci) were used as negative control. (FIG. 5D) The outline of the enzymatic reporter was published elsewhere (Kollmus et al., 1996, Nasim et al., 2000). In brief, translation termination in the internal stop signal generates β-gal protein. In the event of readthrough, a β-gal-luciferase fusion protein will be produced. The ratio of these two proteins was used as the efficiency of readthrough event. For this purpose, two independent constructs were used in our experiment. Construct H8 (pBPLUGA; Kollmus et al., 1996) contains a 17 bp DNA fragment containing an in-frame stop signal. H8A construct (Nasim et al., 2000) is similar to H8 with the exception that it contains a 91 bp DNA fragment. (FIG. 5E) The ratio of gal-luc activity was markedly increased in both constructs following G418 sulphate treatment.

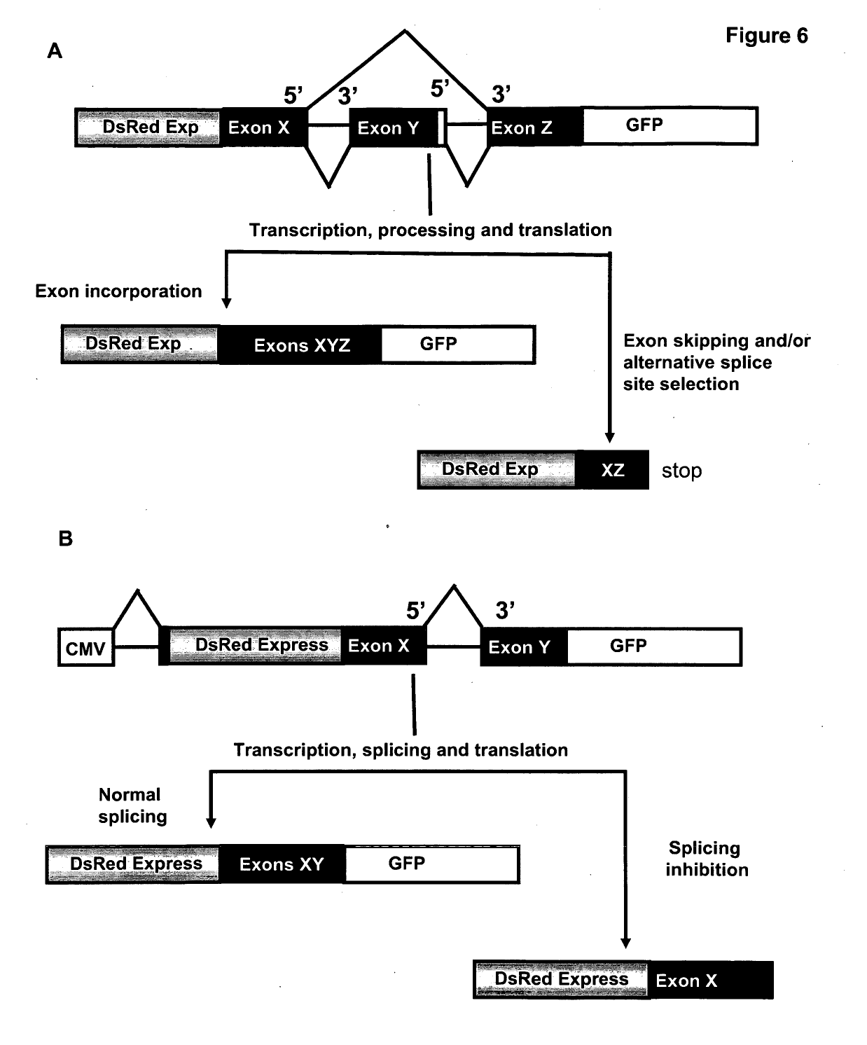

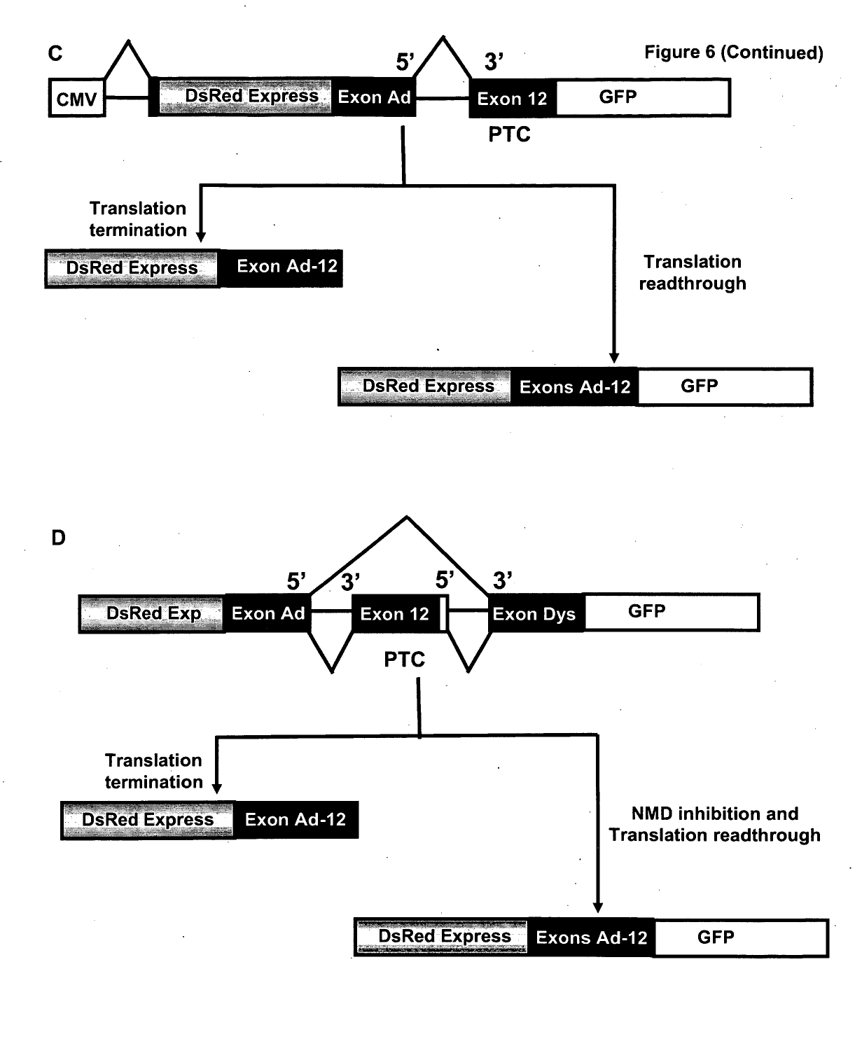

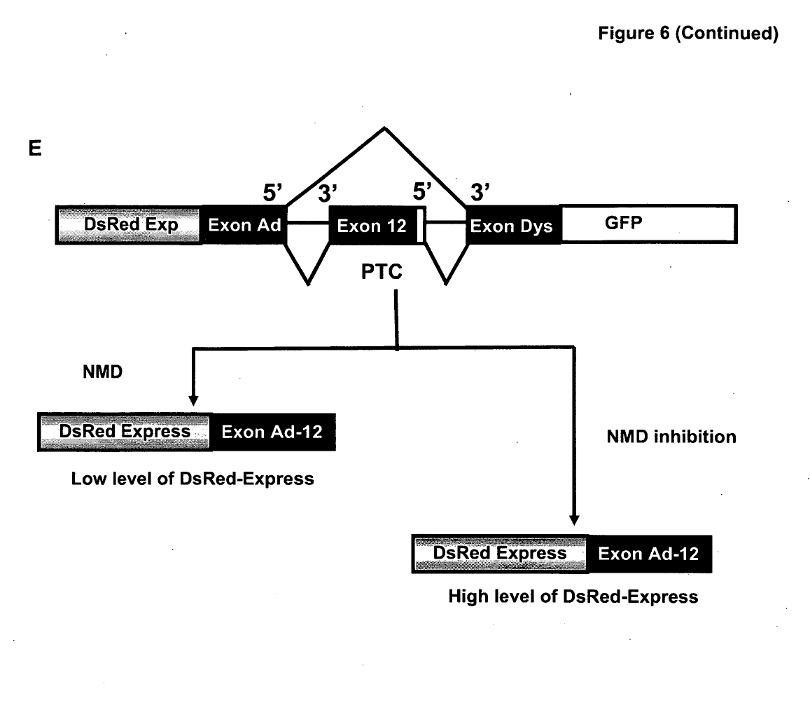

FIG. 6

(A) Dual-reporter in alternative splicing. Underlying principle of this system is described in FIG. 3 with the exception that the middle exon is cloned in such a way that in the event of exon (Y) incorporation, a dual-fluorescence protein will be generated. Exon skipping will generate single fluorescence as the coding sequence of GFP will be out-of-frame with the upstream reporter. The validity of exon skipping vs incorporation can be determined using RT-PCR experiment as described elsewhere (Nasim et al., 2003). A similar principle can also be adapted to determine cryptic splice site selection.

(B) Splicing inhibition. Principle is depicted in FIG. 2. Splicing would generate a dual-fluorescence protein. Factors that inhibit splicing will generate the upstream reporter. These assays (FIGS. 2 and 3) offer an excellent opportunity to isolate effective splicing inhibitors which can be used as anti-viral drugs. In contrast, agents that modulate alternative splicing can be used to engineer replication-competent viruses to treat cancer. A similar principle can also be adapted to isolate factors that enhance splicing. In this case, factors that enhance splicing will generate more dual-fluorescence reporter.

(C) See FIG. 5 legend.

(D) To identify factors that protects nonsense-associated transcript and promote translation read-through. In this case the middle exon should contain a PTC in such that the transcript is decayed by the NMD process. Such reporter will generate low level of upstream protein. Factors that protect the transcript and promote read-through will generate dual-fluorescence reporter. These factors can be used as drug to treat nonsense-associated genetic diseases.

(E) NMD inhibition: Principle is similar to (D). Factors that inhibit the NMD process will generate high level of upstream reporter. These factors can be used to investigate the signaling pathways that affect the NMD process.

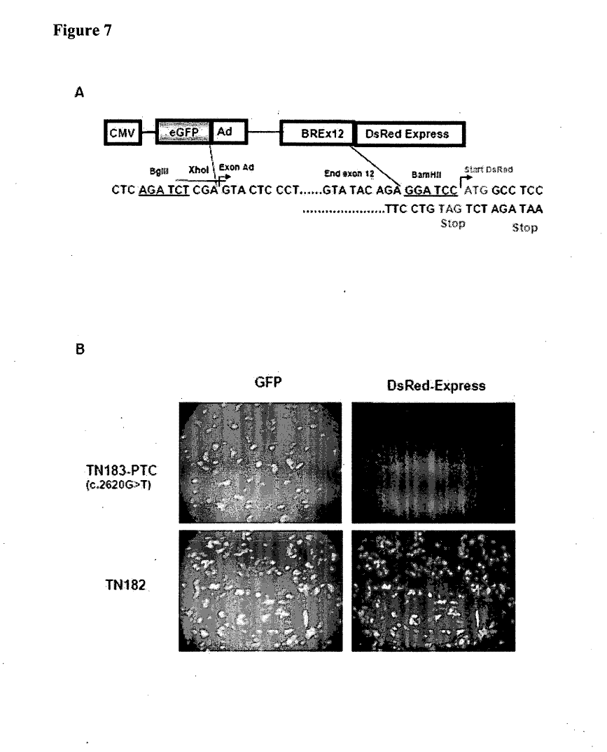

FIG. 7

An outline of the dual-fluorescence reporter. The underlying principle of the reporter is similar to that mentioned above with the exception that the DsRed-express gene is located downstream of the GFP reporter (FIG. 7A). Following transfection the reporter harbouring an in-frame PTC produced only GFP protein (FIG. 7B). The reporter produced both GFP and DsRedExpress proteins following the exchange of the PTC to a sense codon.

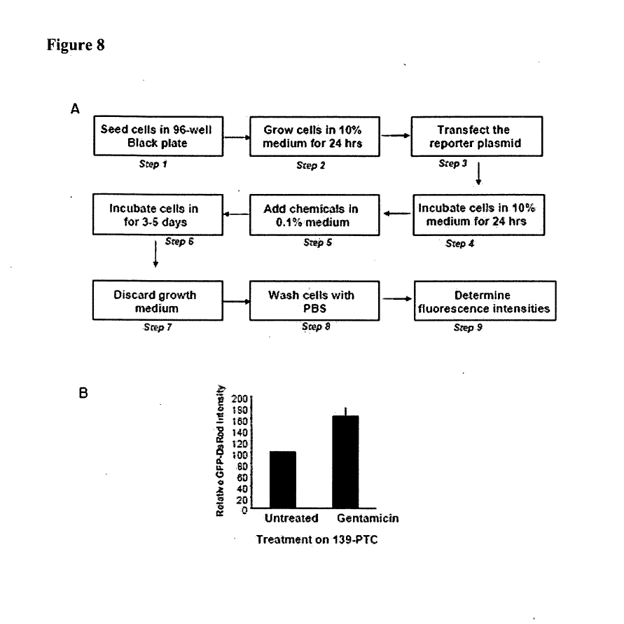

FIG. 8

A step-by-step outline of the translation readthrough assay (FIG. 8A). Cells treated with gentamicin promoted the ratio of GFP and DsRedExpress fluorescence intensities (FIG. 8B).

### DETAILED DESCRIPTION OF THE INVENTION

Splicing

Introns are portions of eukaryotic DNA (eukaryotic genes) which intervene between the coding portions, or “exons,” of that DNA. Introns and exons are transcribed into RNA termed “primary transcript, precursor to mRNA” (or “pre-mRNA”). Introns must be removed from the pre-mRNA so that the native protein encoded by the exons can be produced. The removal of introns from pre-mRNA and subsequent joining of the exons is carried out in the splicing process.

The splicing process is actually a series of reactions, mediated by splicing factors, which is carried out on RNA after transcription but before translation. Thus, a “pre-mRNA” is an RNA which contains both exons and intron(s), and an “mRNA” is an RNA in which the intron(s) have been removed and the exons joined together sequentially so that the protein may be translated therefrom by the ribosomes.

The term “exon,” as used herein has its usual meaning in the art and is a sequence of nucleotides from a gene, which encodes a protein or portions of a protein.

For some embodiments, the exon is exon 12 of BMPR2 (Accession number NM 001294).

For some embodiments, the exon is exon Ad from adenovirus type 2 comprising or consisting of the sequence:

5′gtactccctctcaaaagcgggcatgacttctgcgctaagattgtcag
ttccaaaaacgaggaggatttgatattcaccagctgttggg3′.

For some embodiments, the intron is from adenovirus type 2 comprising or consisting of the sequence:

5′gtgagtcctttgagggtggccgcgtccatctggtcagaaaagacaat
ctttttgttgtcaagcttgctgcacgtctagggcgcagtagtccagggtt
tccttgatgatgtcatacttatcctgtcccttttttttc3′.

For some embodiments, the exon comprises or consists of the last 17 nucleotides of the non-muscle exon 5 of human tropomyosin gene (Accession number NM 153649).

For some embodiments, the intron comprises or consists of the intron of non-muscle exon 5 of human tropomyosin gene (accession number NM 153649).

For some embodiments, the exon Dys is exon 12 of human dystrophin gene (accession number NM 000109) comprising or consisting of the sequence:

5′TTTAACATAGTTTTAATGGATCTCCAGAATCAGAAAGAAAGAGTTGA
ATGACTGGCTAACAAAAACAGAAGAAAGAACAAGGAATGGAGGAAGAGCC
TC3′

For some embodiments, the intron comprises or consists of the last 260 nucleotides of intron 11 of human dystrophin gene (accession number NM 000109).

For some embodiments, the exon is Exon Ad—Exon 12 of BMPR2.

For some embodiments, the exon is Exon Ad—Exon 12 of BMPR2—Exon Dys

For some embodiments, the exon is exon 7 of SMN2 (NM 022877). For some embodiments, the exon is at least exons 6, 7 and 8 of SMN2 (with their corresponding introns).

For some embodiments, the exon is exon 18 of the BRCA2 (NM 000059) gene. For some embodiments, the exon is at least exons 17, 18 and 19 of the BRCA2 gene (with their corresponding introns).

For some embodiments, the exon is exon 2 of the Mcl1 gene (Q07820). For some embodiments, the exon is at least exons 1, 2 and 3 of of the Mcl1 gene (with their corresponding introns).

Suitably, at least one of the exons comprises a genetic variation.

An “intron” as used herein has its usual meaning in the art and is a sequence of nucleotides, which interrupt the protein-coding sequence of a gene. Introns are transcribed into the pre-RNA but are cut out so that they are not translated into protein.

In one embodiment, an intron separates the exons, Exon Ad and Exon 12 of BMPR2.

In one embodiment, introns separate the exons Exon Ad and Exon 12 of BMPR2 and Exon 12 of BMPR2 and Exon 12 of dystrophin gene.

In one embodiment, the intron comprises one or more (translation) stop codons—such as two or three or more stop codons.

In another embodiment, the intron does not comprise one or more (translation) stop codons—such as two or three or more stop codons.

In one embodiment, the constructs described herein comprise one or more—such as two or three or more—(translation) stop signals downstream of the first exon. This may prevent the production of dual-reporter signals in the event of inefficient splicing.

In one embodiment, the intron, the downstream exon and the downstream reporter may be cloned in such a way that retention of the intron disrupts the reading frame (eg. It creates a frameshift). The disruption (eg. one or more mutations) should commence preferably in the intron, and/or in the downstream exon and/or at the beginning of the downstream reporter.

Splicing Unit

As used herein the term “splicing unit” refers to at least two exons and at least one intron with the at least one intron separating the at least two exons such that the splicing event results in removal of the intron.

As will be appreciated by the skilled person, the splicing unit in the context of the present invention may comprise the minimal exon and intron sequences that are required in order for a splicing unit to be formed. Accordingly, fragments of exons and introns are encompassed by the present invention as long as the splicing event results in removal of the intron(s).

The splicing unit may be a naturally occurring splicing unit.

The splicing unit may be a non-naturally occurring splicing unit.

The splicing unit may be a hybrid splicing unit comprising one or more exons or one or more introns from a different gene sequence from the same or a different genome.

The splicing unit may be a recombinant splicing unit.

For some embodiments, the splicing unit comprises exons from exon Ad and/or exon 12 of BMPR2 and/or exon Dys (exon 12 of dystrophin gene) from HC 9 (Nasim et al., 2003), wherein exon Ad and exon 12 of BMPR2 and/or exon 12 of BMPR2 and exon Dys from HC 9 are separated by introns.

For some embodiments, the splicing unit comprises exons in the 5′ to 3′ direction from exon Ad and exon 12 of BMPR2, wherein exon Ad and exon 12 of BMPR2 are separated by an intron.

For some embodiments, the splicing unit comprises exons in the 5′ to 3′ direction from exon Ad to exon 12 of BMPR2 to Dys from HC 9, wherein exon Ad and exon 12 of BMPR2 are separated by an intron and wherein exon 12 of BMPR2 and exon Dys from HC 9 are separated by an intron.

As used herein, the term “first exon” refers to the exon adjacent to the 3′ end of the first reporter that is adjacent to the promoter.

As used herein, the term “second exon” refers to the exon adjacent to the 5′ end of the second reporter that is separated from the first reporter by the splicing unit.

As used herein, the term “intermediate exon” refers to the exon that is located between the first exon and the second exon and is separated from the first and second exons by intron sequences.

At least one of the exons in the splicing unit may comprise one or more genetic variations.

Genetic Variation

As described herein, methods are provided that can be used to determine the effect of a genetic variation on the integrity of an RNA transcript or RNA metabolism. To determine if a genome sequence variation affects RNA transcript integrity or RNA metabolism, the effect of the mutation can be characterized using the methods described herein. Whilst the PTC bearing transcripts may be degraded by nonsense-mediated decay, they have also been reported to inhibit splicing and cause altered splice site selection in pre-mRNAs. Functional characterization of sequence variations thus relies, at least in part, on understanding whether the variation activates splicing (eg. aberrant splicing) or triggers the NMD pathway.

A nucleic acid construct as described herein is provided (typically as part of a vector or plasmid) and transfected into a cell—such as a mammalian cell. Depending on the extent of reporter activity—such as the ratio of the two reporter activities—it is possible to determine the effect that the genetic variation has on the RNA transcript.

Referring to FIGS. 2 and 6C, using the construct comprising at least two different reporters separated by an in frame splicing unit, wherein said splicing unit comprises at least two exons separated by at least one intron and wherein at least one exon comprises the genetic variation. In one embodiment, the variation is introduced into the upstream and/or the downstream exon. Expression of both reporters in the cell is indicative that said mutation is not a PTC and that aberrant splicing occurs. Expression of a single reporter upstream of the splicing unit in combination with each of the exons is indicative that said mutation is a PTC that causes translational termination. Accordingly, it can be deduced whether or not the mutation causes premature termination of translation.

For some embodiments, it may be desirable to further verify the nature of the RNA transcript. In this regard, various methods in the art can be used—such as RT-PCR. Expression of both reporters in the cell will typically result in the transcription and splicing of the upstream and downstream reporters and at least two exons. Expression of the single reporter in the cell will typically result in the transcription, splicing and translation of the upstream reporter and at least two exons, wherein the exon containing the genetic variation is not translated downstream of the genetic variation. Accordingly, the exon downstream of the genetic variation will not be translated and nor will the downstream reporter.

Referring to FIG. 3, using the construct comprising at least two different reporters separated by an in frame splicing unit, wherein said splicing unit comprises at least three exons and two introns, wherein at least one exon (eg. the exon separated by the two introns) comprises the genetic variation expression of both reporters and all three exons in the cell is indicative that said genetic variation is not a PTC and results in exon incorporation. The expression of both reporters and two exons without expression of the exon comprising the genetic variation is indicative that said mutation is not a PTC and causes exon skipping. Expression of a single reporter upstream of the splicing unit in combination with the first two introns downstream of said reporter is indicative that said genetic variation is a PTC and that the RNA transcript is subject to nonsense mediated decay. Accordingly, it can be deduced whether or not the mutation causes exon incorporation, exon skipping or whether the mutation is a PTC that results in nonsense medicated decay of the RNA transcript.

For some embodiments, it may be desirable to further verify the nature of the RNA transcript. In particular, it may be desirable to distinguish between exon incorporation and exon skipping. Since both events result in dual reporter expression, the identity of the transcript may be further determined using methods in the art—such as RT-PCR.

Accordingly, using the methodology described herein it is possible to determine how a genome sequence variation—such as a PTC—affects RNA transcript integrity or RNA metabolism. Genetic variations that affect RNA transcript integrity or RNA metabolism may provide, for example, insights into their involvement in disease. The genetic variations may also be of use in the assay methods described herein in which it is desirable to create a system in which a known genetic variation has a known affect on RNA transcript integrity or RNA metabolism such that agents can be identified that modulate this affect.

The genetic variation may be a mutation. The genetic variation may be a mutation that does or does not incorporate a PTC into the RNA transcript.

Mutation

As used herein, the term “mutation” refers to any mutation that effects the integrity of an RNA transcript or effects RNA metabolism and includes, but is into limited to, a nonsense mutation, an insertion mutation, a deletion mutation or a substitution mutation.

In one embodiment, at least one mutation is incorporated into at least one or at least two or at least three exons.

In one embodiment, at least one mutation is incorporated into the upstream and/or the downstream and/or the intermediate exon.

In one embodiment, the exon into which a mutation is incorporated is exon 12 of BMPR2.

In one embodiment, the exon may comprise a naturally occurring mutation (which may incorporate a PTC) and which causes exon 7 skipping of SMN2 or exon 18 skipping of BRCA2 or exon 2 skipping of Mcl1.

The mutation may be a nonsense mutation (eg. a nonsense codon). The nonsense mutation may incorporate a PTC such that in the event of read-through a dual fluorescence typically occurs and in the event of translation termination only the upstream reporter is expressed.

For some embodiments, one or more nonsense mutations can be included in exon 12 of BMPR2 which will be recognized as a translation stop signal. This stop signal may translational read-through and/or splicing and/or NMD. Accordingly, this exon can be used, for example, in assay systems to identify agents that modulate translational read-through and/or splicing and/or NMD. Accordingly, the one or more nonsense codons in exon 12 of BMPR2 can be used, for example, in assay systems to identifying agents that modulate these events.

In one embodiment, the mutation is c.2292insA in exon 12 of BMPR2. In one embodiment, the mutation is c.2386delG in exon 12 of BMPR2. In one embodiment, the mutation is c.2695C>T in exon 12 of BMPR2. In one embodiment, the mutation is c.2386delG in exon 12 of BMPR2. In one embodiment, the mutation is c.2620G>T in exon 12 of BMPR2.

In another embodiment, the PTC is selected from the group consisting of mutation c.2292insA in exon 12 of BMPR2; mutation c.2386delG in exon 12 of BMPR2; mutation c.2695C>T in exon 12 of BMPR2; mutation c.2386delG in exon 12 of BMPR2; mutation is c.2620G>T in exon 12 of BMPR2. Each of these mutations is recognized as a PTC and thereby cause termination of translation. Accordingly, each of these mutations can be used in an assay to identify one or more agents that modulate, for example, translational read-through and/or splicing and/or NMD.

Construct

The term “construct” is synonymous with terms such as “conjugate” and “cassette”. Typically, the nucleic acid construct will form part of a plasmid or a vector—such as an expression vector.

There are described herein, nucleic acid constructs comprising at least two reporters separated by an in frame splicing unit. The reporters will be in-frame with a promoter such that expression from the promoter results in expression of each of the reporters, unless transcription is terminated with the splicing unit such that the at least second reporter is not expressed or substantially not expressed. The promoter may form part of the construct or it may form part of the vector. The promoter will be at the 5′ end of the nucleic acid construct such that expression of some or all of the reporters and/or part or all of the splicing unit occurs.

Suitably, the promoter is operably linked to the one or more reporters and the one or more splicing units.

In one embodiment, the in frame splicing unit comprises a first and a second exon separated by an intron sequence, wherein the intron does not comprise a stop codon.

In one embodiment, the in frame splicing unit comprises a first and a second exon separated by an intron sequence, wherein the first and/or second exon comprises a genetic variation.

In another embodiment, the in frame splicing unit comprises a first, a second and an intermediate exon, wherein the intermediate exon is separated from the first and second exons by intron sequences, and wherein the first and/or second and/or intermediate exon comprises a genetic variation.

In one embodiment, the intron sequence(s) do not include one or more translation stop signals—such as an in-frame translation stop signal(s).

In one embodiment, the intron sequence(s) do not include three translation stop signals—such as an in-frame translation stop signal(s).

As used herein, the term “upstream” refers to the 5′ direction of the nucleic acid construct. Thus, by way of example, referring to FIG. 2, the DsRedExpress reporter is located upstream of Exon Ad.

As used herein, the term “downstream” refers to the 3′ direction of the nucleic acid construct. Thus, by way of example, referring to FIG. 2, the Exon Ad is located downstream of the DsRedExpress reporter.

Reporter

As described herein, the nucleic acid construct comprises at least two reporters. Suitably, each reporter will result in a detectable signal that is distinguishable from the other.

A wide variety of reporters may be used with preferred reporters providing conveniently detectable signals (eg. by spectroscopy or by florescence). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. The reporter genes may be any reporter genes that are known in the art including β-galactosidase, β-glucuronidase, luciferase and/or fluorescent protein.

In one embodiment, the reporter(s) is a fluorescent protein.

Where two or more reporters are used, the reporters may be the same or different reporters.

Where two or more reporters are used, the reporters may be the same or different fluorescent reporters.

Where two or more reporters are used, the reporters are typically different reporters (eg. different reporters of the same type or different reporters of a different type) such that the expression of each individual reporter can be determined.

Where two florescent reporters are used, the reporters are typically different florescent reporters with different emission spectra such that the expression of each individual florescent reporter can be determined. Suitably, the expression of each individual florescent reporter should be easily distinguishable under a fluorescence microscope.

In one embodiment, the fluorescent protein is green fluorescent protein (GFP) or a protein derived from GFP. Wild type GFP from the jellyfish Aequorea victoria has a major excitation peak at a wavelength of 395 nm and a minor one at 475 nm. The fluorescence emission peak of GFP is at 509 nm which is in the lower green portion of the visible spectrum, hence the name. The GFP-like protein from the sea pansy (Renilla reniformis) has a single major excitation peak at 498 nm.

Modified forms of GFP can also be used. Modified GFPs include mutants with increased fluorescence and/or in which the major excitation peak has been shifted, for example, to 490 nm with the peak emission kept at 509 nm. Modified GFPs can also include mutants with a shifted emission peak. Thus, colour mutants are suitable, including the cyan fluorescent protein and the yellow fluorescent protein.

The term “mutant form of GFP” as used herein denotes fluorescent proteins that are GFP-like including, without limitation, R. reniformis GFP-like protein, synthetic mutants of wild-type GFP, modified GFP, and colour mutants, in that the properly folded proteins have generally similar fluorescence excitation/emission spectra and misfolded proteins are substantially not fluorescent.

For some embodiments, at least one of the reporters (such as the reporter adjacent to the promoter) is DsRedExpress, is derived from DsRedExpress or is a mutant of DsRedExpres.

For some embodiments, at least one of the reporters is GFP, derived from GFP or is a mutant form of GFP.

For some embodiments, the upstream reporter is DsRedExpress, is derived from DsRedExpress or is a mutant of DsRedExpress and the downstream reporter is GFP, derived from GFP or is a mutant form of GFP. For other embodiments, the upstream reporter is GFP, derived from GFP or is a mutant form of GFP and the downstream reporter is DsRedExpress, is derived from DsRedExpress or is a mutant of DsRedExpress.

The level of expression of the reporter may be determined by various methods that are known in the art—such as microscopy, florescence microscopy, flow cytometry and FACS analysis.

In one embodiment, ratios of reporter activity may be measured and compared—such as the ratio of reporter activity in the presence and absence of an agent.

Assays

Appreciation of the mechanisms that influence the integrity of an RNA transcript or RNA metabolism may promote the development of therapeutic strategies that can be applied to broad classes of human diseases. The assays described herein may prove to be useful to study a wide range of diseases caused by or associated with defects in the integrity of an RNA transcript or RNA metabolism. Since the assays described herein are suitable for high-throughput screening they will be useful for identifying agents that modulate the integrity of an RNA transcript or RNA metabolism.

A cell can be transfected with a nucleic acid construct as described herein, such that expression of the construct results in the defect in RNA metabolism that it to be investigated. The cell can then be used in an assay—such as a high throughput assay—to identify one or more agents that modulate the defect in RNA metabolism by monitoring the expression (eg. the ratio of expression) of the reporters present on the nucleic acid construct.

The agents that modulate the defect in RNA transcription or metabolism may be useful in the treatment and/or prevention of disease—such as human disease.

The assays may be a screen, whereby a number of agents (e.g. one or more, two or more, three or more, four or more etc) are tested simultaneously, sequentially or individually.

It is expected that the assays will be suitable for both small and large-scale screening of test compounds.

Suitably, the assays are cell based assays. Suitably, the cell based assays are single cell based assays. Suitably, the cell based assays are mammalian cell based assays. Suitably, the cell based assay are single mammalian cell based assays.

The assays described herein may be used to study early stages of developing embryos—such as the integrity and efficiency of splicing in the early stages of developing embryos.

Modulation of Splicing

The assays described herein may be used to study a wide range of alternatively spliced genes linked to diseases. Since the assays described herein are suitable for high-throughput screening they will be useful for identifying agents that affect splicing reactions.

A cell can be transfected with a nucleic acid construct in which expression of the construct results in incomplete/incorrect splicing. It is then possible to use the cell in an assay—such as a high throughput assay—to identify one or more agents that modulate the incomplete/incorrect splicing by monitoring the expression (eg. the ratio of expression) of the reporters.

By way of example, the assays described herein may be used to identify agents that are of use in treating human disease—such as spinal muscular atrophy (SMA). SMA is caused by homogygous loss of survival motor neuron gene (SMN1). SMN2, a nearly identical copy of SMN1, is preserved in SMA patients. A single nucleotide difference between these two genes causes exon 7 skipping of SMN2, produces a non-functional protein and thus cannot compensate the loss of SMN1. Drugs or transacting factors that could increase the incorporation of exon 7 of SMN2 would provide protection from the disease. The assays described herein may be useful to identify agents—such as trans-acting factors that sufficiently incorporate exon 7 of SMN2.

Accordingly, in one embodiment, the exon is exon 7 of SMN2. This exon may be used in combination with, for example, exon Ad and exon Dys in order to obtain a rapid insight into the assay system. For some embodiments, at least exons 6 and 8 of SMN2 (with their corresponding introns) may additionally be used in order to refine the assay system.

By way of example, the assays described herein may be used to identify agents that modulate skipping of exon 18 of the BRCA2 gene. Skipping of exon 18 of the BRCA2 gene is associated with breast cancer (Fackenthal et al., 2002 Am J Hum Genet 71:625-31). Akin to SMN2, factors that incorporate the exon 18 of BRCA2 might provide a protection or attenuate disease progression.

Accordingly, in one embodiment, the exon is exon 18 of the BRCA2 gene. This exon may be used in combination with, for example, exon Ad and exon Dys in order to obtain a rapid insight into the assay system. For some embodiments, at least exons 16 and 17 of the BRCA2 gene (with their corresponding introns) may additionally be used in order to refine the assay system.

By way of further example, the assays described herein may be used to identify agents that modulate skipping of exon 2 of the Mcl1 gene. This results in a shorter protein that causes cells to die rather than remain alive (Bae et al., 2000, J Biol Chem 275: 25255-61). Hence, factors that modulate alternative splicing of genes involved in promoting or inhibiting apoptosis may have therapeutic potential in treating cancer.

Accordingly, in one embodiment, the exon is exon 2 of the Mcl1 gene. This exon may be used in combination with, for example, exon Ad and exon Dys in order to obtain a rapid insight into the assay system. For some embodiments, at least exons 1 and 3 of the Mcl1 gene (with their corresponding introns) may additionally be used in order to refine the assay system.

Referring to FIG. 6B, a nucleic acid construct comprising at least two different reporters separated by an in frame splicing unit, wherein said splicing unit comprises at least two exons separated by at least one intron and wherein the at least one intron does contain stop codon(s) is used to identify an agent that modulates splicing. A difference in reporter activity in comparison to a cell that has not been exposed to the at least one agent is indicative that said agent modulates splicing. In further detail, expression of both reporters in the cell is indicative of normal splicing. The expression of a single reporter in the cell is indicative of splicing inhibition. Expression of both reporters in the cell exposed to the agent and the expression of a single reporter in the cell not exposed to the agent is indicative that said agent promotes normal splicing. An increase in the expression of both reporters in the cell exposed to the agent in comparison to the cell not exposed to the agent is indicative that said agent promotes normal splicing. Expression of a single reporter in the cell exposed to the agent and the expression of both reporters in the cell not exposed to the agent is indicative that said agent promotes splicing inhibition. An increase in the expression of the single reporter in the cell exposed to the agent in comparison to the cell not exposed to the agent is indicative that said agent promotes splicing inhibition.

Suitably, the RNA transcripts can be further verified using methods such as RT-PCR. Identification of a transcript comprising the at least two different reporters separated by the at least two exons confirms that normal splicing has occurred. Identification of transcript comprising the only the upstream reporter and all or part of the exon adjacent to the upstream reporter is indicative of splicing inhibition.

Modulation of Translation & Translation Read-Through

The assays described herein can also be used to identify agents that modulate translation read-through. The majority of PTC bearing mutations identified in human diseases follow the NMD pathway and as a result produce insufficient levels of full-length protein.

Intriguing attempts have been made to treat genetic disorders resulting from PTCs with aminoglycosides and small molecules (PTC124) aimed at promoting nonsense codon read-through. The read-through efficiencies achieved by aminoglycosides and PTC124 are as low as 2-10%. This offers the opportunity for identification of more efficient factors. The major drawback for the identification of such factors using these systems is that the assay can not be measured directly on intact cells and requires cell wall disruption or addition of a substrate and hence offers a limited opportunity for high-throughput screening. In contrast, the assays described herein may be used to identify factors that promote translation readthrough in living cells. In the event of read-through, a fusion dual-fluorescence protein (eg. DsRed Express and GFP) will typically be produced, whilst translation termination produces only, for example, DsRed-Express protein. Thus the ratio of two reporter intensities determines the efficiency of read-through.

Since the dual-fluorescence assay is suitable for high-throughput screening of small molecules, peptide and nucleic acids, it may be useful in isolation of factors that achieve the maximum read-through efficiency. Hits derived from a screen can be further validated using a luminescence based reporter (13).

Referring to FIGS. 2 and 5, a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon is provided. A difference in reporter activity in a cell comprising the nucleic acid construct that is exposed to the agent in comparison to a cell that has not been exposed to the agent is indicative that said agent modulates translation. In further detail, expression of both reporters in the cell is indicative of translation read-through. Expression of a single reporter in the cell is indicative of translation termination. Expression of both reporters in the cell exposed to the agent and the expression of a single reporter in the cell not exposed to the agent is indicative that said agent promotes translational read-through. An increase in the expression of both reporters (ratio) in the cell exposed to the agent in comparison to the cell not exposed to the agent is indicative that said agent promotes translation read-through. Expression of a single reporter in the cell exposed to the agent and the expression of both reporters in the cell not exposed to the agent is indicative that said agent promotes translation termination. An increase in the expression of the single reporter in the cell exposed to the agent in comparison to the cell not exposed to the agent is indicative that said agent promotes translation termination.

Suitably, the RNA transcripts can be further verified using methods such as RT-PCR. Identification of a transcript comprising the at least two different reporters separated by the at least two exons confirms that translational read-though has occurred. Identification of a transcript comprising only the upstream reporter and only the exons upstream of the PTC is indicative of translation termination.

Suitably, the premature termination codon is encoded by the insertion c.2292insA, the deletion c.2386delG or the nonsense mutation c.2695C>T in exon 12 of BMPR2. Each of these mutations results in the incorporation of a premature termination codon. Accordingly, the nucleic acid construct comprising these mutations can be used to assay for agents that modulate translation termination. Accordingly, there is also provided a method for identifying an agent that modulates translation termination comprising the steps of: (a) providing a nucleic acid construct comprising at least two different reporters separated by an in frame splicing unit, wherein said splicing unit comprises at least two exons separated by at least one intron and wherein at least one exon (eg. the exon downstream of said intron) comprises one of the mutations that are incorporated as a premature termination codon; (b) transfecting said construct into a cell; (c) culturing said cell; (d) contacting said cell with the at least one agent; and (e) determining the effect of said agent on translation termination, wherein a difference in reporter activity in comparison to a cell that has not been exposed to the at least one agent is indicative that said agent modulates translation termination. An increase in the expression of the single reporter in the cell exposed to the agent in comparison to the cell not exposed to the agent is indicative that said agent promotes translation termination. A decrease in the expression of the single reporter in the cell exposed to the agent in comparison to the cell not exposed to the agent is indicative that said agent promotes translation termination.

In one embodiment, the exon comprising the PTC is exon 7 of SMN2 as described herein.

In one embodiment, the exon comprising the PTC is exon 18 of the BRCA2 gene as described herein.

In one embodiment, the exon comprising the PTC is exon 2 of the Mcl1 gene as described herein.

Modulation of Translation and/or Nonsense Mediated Decay

The assay systems described herein also offer the possibility for identifying agents that can be used to treat disease based on approaches that specifically inhibit disease associated NMD transcripts. An advantage of assays described herein is that the system based on a reporter—such as DsRed-Express—by comparison to GFP-based approaches encounters much reduced autofluorescence. The single cell based system is suitable for high-throughput screening.

Referring to FIG. 6D, a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon is provided A difference in reporter activity in a cell comprising the nucleic acid construct that is exposed to the agent in comparison to a cell that has not been exposed to the agent is indicative that said agent modulates translation and/or nonsense mediated decay. In further detail, expression of both reporters in the cell is indicative of translation read-through and NMD inhibition. Expression of a single reporter in the cell is indicative of translation termination. Expression of both reporters in the cell exposed to the agent and the expression of a single reporter in the cell not exposed to the agent is indicative that said agent promotes translational read-through and inhibits nonsense mediated decay. An increase in the expression of both reporters in the cell exposed to the agent in comparison to the cell not exposed to the agent is indicative that said agent promotes translational read-through and inhibits nonsense mediated decay. Expression of a single reporter in the cell exposed to the agent and the expression of both reporters in the cell not exposed to the agent is indicative that said agent promotes translation termination. An increase in the expression of the single reporter in the cell exposed to the agent in comparison to the cell not exposed to the agent is indicative that said agent promotes translation termination.

Suitably, the RNA transcripts can be further verified using methods such as RT-PCR. Identification of a transcript comprising the at least two different reporters separated by the at least two exons upstream of the PTC confirms that translational read-though and NMD inhibition has occurred. Identification of transcript comprising only the upstream reporter and only the exons upstream of the PTC is indicative of translation termination.

Suitably, the premature termination codon is encoded by the insertion c.2292insA, the deletion c.2386delG or the nonsense mutation c.2695C>T in exon 12 of BMPR2. Each of these mutations results in the incorporation of a premature termination codon. Accordingly, the nucleic acid construct comprising these mutations can be used to assay for agents that modulate translation termination and in which the transcript is subject to NMD. Accordingly, there is provided a method for identifying an agent that modulates translation termination and nonsense mediated decay comprising the steps of: (a) providing a nucleic acid construct comprising at least two different reporters separated by an in frame splicing unit, wherein said splicing unit comprises at least two exons separated by at least one intron and wherein at least one exon (eg. the exon downstream of said intron) comprises the mutations that results in the incorporation of a premature termination codon; (b) transfecting said construct into a cell; (c) culturing said cell; (d) contacting said cell with at least one agent; and (e) determining the effect of said agent on translation termination and nonsense mediated decay; wherein a difference in reporter activity in comparison to a cell that has not been exposed to the at least one agent is indicative that said agent modulates translation termination and nonsense mediated decay. An increase in the expression of the single reporter in the cell exposed to the agent in comparison to the cell not exposed to the agent is indicative that said agent promotes translation termination and nonsense mediated decay. A decrease in the expression of the single reporter in the cell exposed to the agent in comparison to the cell not exposed to the agent is indicative that said agent inhibits translation termination and nonsense mediated decay.

Modulation of Exon Skipping and/or Exon Incorporation

Referring to FIG. 6A, a nucleic acid construct comprising at least two different reporters separated by an in frame splicing unit, wherein said splicing unit comprises at least three exons separated by at least two introns is provided for identifying at least one agent that modulates exon incorporation or exon skipping. A difference in reporter activity in a cell comprising the nucleic acid construct that is exposed to the agent in comparison to a cell that has not been exposed to the at least one agent is indicative that said agent modulates exon skipping or exon incorporation. In further detail, expression of both reporters in the cell is indicative of exon incorporation. Expression of a single reporter in the cell is indicative of exon skipping and/or alternative splice site selection. Expression of both reporters in the cell exposed to the agent and the expression of a single reporter in the cell not exposed to the agent is indicative that said agent promotes exon incorporation. An increase in the expression of both reporters in the cell exposed to the agent in comparison to the cell not exposed to the agent is indicative that said agent promotes exon incorporation. Expression of a single reporter in the cell exposed to the agent and the expression of both reporters in the cell not exposed to the agent is indicative that said agent promotes exon skipping and/or alternative splice site selection. An increase in the expression of the single reporter in the cell exposed to the agent in comparison to the cell not exposed to the agent is indicative that said agent promotes exon skipping and/or alternative splice site selection.

Suitably, the RNA transcripts can be further verified using methods such as RT-PCR. Identification of a transcript comprising the at least two different reporters separated by the at least three exons confirms that exon incorporation has occurred. Identification of transcript comprising only the upstream reporter and only the first two exons downstream of the reporter is indicative of exon skipping and/or alternative splice site selection.

Modulation of Exon Skipping and/or Exon Incorporation and/or NMD

Referring to FIG. 3, a nucleic acid construct comprising two different reporters separated by an in frame splicing unit, wherein said splicing unit comprises three exons separated by two introns, wherein at least one exon (eg. the exon separated by the two introns) comprises a premature termination codon is provided for identifying at least one agent that modulates exon incorporation and/or exon skipping and/or nonsense mediated decay. A difference in reporter activity in a cell comprising the nucleic acid construct that is exposed to the agent in comparison to a cell that has not been exposed to the agent is indicative that said agent modulates exon incorporation or exon skipping or nonsense mediated decay. In further detail, expression of both reporters in the cell is indicative of exon incorporation or exon skipping. Expression of a single reporter in the cell is indicative of nonsense mediate decay. Expression of both reporters in the cell exposed to the agent and the expression of a single reporter in the cell not exposed to the agent is indicative that said agent promotes exon incorporation or exon skipping. An increase in the expression of both reporters in the cell exposed to the agent in comparison to the cell not exposed to the agent is indicative that said agent promotes exon incorporation or exon skipping. Expression of a single reporter in the cell exposed to the agent and the expression of both reporters in the cell not exposed to the agent is indicative that said agent promotes nonsense mediated decay. An increase in the expression of the single reporter in the cell exposed to the agent in comparison to the cell not exposed to the agent is indicative that said agent promotes nonsense mediated decay.

Suitably, the RNA transcripts can be further verified using methods such as RT-PCR. Identification of a transcript comprising the at least two different reporters separated by the at least three exons confirms that exon incorporation has occurred. Identification of a transcript comprising the at least two different reporters separated by exon adjacent to the upstream reporter and the exon adjacent to the downstream reporter is indicative that exon skipping has occurred. Identification of a transcript comprising only the upstream reporter and only the exons upstream of the PTC is indicative of translation termination and nonsense mediate decay.

In one embodiment, the exon comprising the PTC is exon 7 of SMN2 as described herein.

In one embodiment, the exon comprising the PTC is exon 18 of the BRCA2 gene as described herein.

In one embodiment, the exon comprising the PTC is exon 2 of the Mcl1 gene as described herein.

Modulation of Nonsense Mediated Decay

As described herein, the assay systems also offer the possibility for innovative and useful treatments based on approaches specifically to inhibit disease associated NMD transcripts.

Referring to FIG. 6E, a nucleic acid construct is provided comprising two different reporters separated by an in frame splicing unit, wherein said splicing unit comprises three exons separated by two introns, wherein at least one exon (eg. the exon separated by the two introns) comprises a premature termination codon. A difference in reporter activity in a cell comprising the nucleic acid construct that has been exposed to the agent in comparison to a cell that has not been exposed to the at least one agent is indicative that said agent modulates nonsense mediated decay. A low level of the reporter upstream of the splicing unit in the cell exposed to the at least one agent in comparison to the cell that has not been exposed to the at least one agent is indicative that said agent promotes nonsense mediated decay. A high level of the reporter upstream of the splicing unit in the cell exposed to the at least one agent in comparison to the cell that has not been exposed to the at least one agent is indicative that said agent inhibits nonsense mediated decay.

Suitably, the “level” of the reporter refers to the intensity of the reporter compared to the control transcript—such as wild type reporter—which is not subject to NMD.

Suitably, a “low level” means that the transcript is decayed by the NMD machinery and hence will produce insufficient protein.

Suitably, a “high level” means that the transcript is not decayed by the NMD machinery and sufficient protein is produced.

For some embodiments, it is desirable to verify that a genetic variation—such as a genetic variation containing a PTC—is subject to NMD. A number of methods in the art can be used to determine if a genetic variation is subject to NMD—such as quantitative RT-PCR. In one specific example, a double intron splicing unit containing one or mutations to be tested are introduced at the 3 ′end of a β-galactosidase gene in such a way that the incorporation of the PTC would lead to the truncation of a full-length protein. Thus, if the mRNA is subjected to NMD, inhibition of translation using cycloheximide or puromycin would lead to the increased level of PTC bearing mRNA and therefore the level of the truncated protein would also be increased.

For further confirmation that a mutation is subject to NMD, plasmids encoding siRNA that inhibit expression of hUpf1 (9) can be transfected together with the reporter plasmid. Compared with cells transfected with the reporter alone, the gal-luc ratio will increase between 3 and 6 fold when hUpf1 RNA is knocked down by RNAi.

Suitably, the premature termination codon is encoded by the insertion c.2292insA, the deletion c.2386delG or the nonsense mutation c.2695C>T in exon 12 of BMPR2. Each of these mutations results in the incorporation of a premature termination codon. Accordingly, there is provided in a further aspect, a method for identifying at least one agent that modulates nonsense mediated decay comprising the steps of: (a) providing a nucleic acid construct comprising two different reporters separated by an in frame splicing unit, wherein said splicing unit comprises three exons separated by two introns, wherein at least one exon (eg. the exon separated by the two introns) comprises the mutations that results in the incorporation of the premature termination codon; (b) transfecting said construct into a cell; (c) culturing said cell; (d) contacting said cell with at least one agent; and (e) determining the effect of said agent on nonsense mediated decay; wherein a difference in reporter activity in comparison to a cell that has not been exposed to the at least one agent is indicative that said agent modulates nonsense mediated decay. A low level of the reporter upstream of the splicing unit in the cell exposed to the at least one agent in comparison to the cell that has not been exposed to the at least one agent is indicative that said agent promotes nonsense mediated decay. A high level of the reporter upstream of the splicing unit in the cell exposed to the at least one agent in comparison to the cell that has not been exposed to the at least one agent is indicative that said agent inhibits nonsense mediated decay.

Cell

The nucleic acid construct (typically inserted into a vector) may be transfected or transformed into a cell (eg. a host cell) in order to express the nucleic acid construct in the compatible cell when cultured under appropriate conditions which bring about expression.

The vector may be recovered from the cell.

A further embodiment of the present invention provides host cells transformed or transfected with the nucleic acid construct or a vector comprising the nucleic acid construct. The cells will be chosen to be compatible with the said vector and may for example be prokaryotic (for example bacterial), fungal, yeast, plant or mammalian cells.

Suitably, the cell is a mammalian cell.

Vector

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species.

Vectors may be transformed into a suitable host cell as described herein.

The vectors may be for example, plasmid, virus or phage vectors provided with an origin of replication, optionally a promoter for the expression of the polynucleotide and optionally a regulator of the promoter.

Vectors may contain one or more selectable marker genes.

The vectors may be used in vitro.

Nucleotide Sequence

Aspects of the present invention may involve the use of nucleotide sequences, which are available in databases.

As used herein, the term “nucleotide sequence” is synonymous with the term “polynucleotide”.

The nucleotide sequence may be DNA or RNA of genomic or synthetic or recombinant origin. The nucleotide sequence may be double-stranded or single-stranded whether representing the sense or antisense strand or combinations thereof.

The nucleotide sequence may be DNA.

The nucleotide sequence may be prepared by use of recombinant DNA techniques (e.g. recombinant DNA).

The nucleotide sequence may be cDNA.

The nucleotide sequence may be the same as the naturally occurring form, or may be derived therefrom.

Agent

As used herein, the term “agent” may be a single entity or it may be a combination of entities.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule (eg. DNA, cDNA, RNA or siRNA)—which may be a sense or an anti-sense molecule. The agent may even be an antibody.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptide synthesised synthetically (such as, by way of example, either using a peptide synthesizer or by recombinant techniques or combinations thereof), a recombinant agent, an antibody, a natural or a non-natural agent or a fusion protein.

Typically, the agent will be an organic compound. Typically the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent may comprise at least the one of said cyclic groups linked to another hydrocarbyl group.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

The agent may be siRNA (Fire A et al. (1998), Nature 391: 806-811). The siRNA may comprise partially purified RNA, substantially pure RNA, synthetic RNA, or recombinantly produced RNA, as well as altered RNA that differs from naturally-occurring RNA by the addition, deletion, substitution and/or modification of one or more nucleotides. One or both strands of the siRNA may comprise a 3′ overhang. In order to enhance the stability of the siRNA, the 3′ overhangs may be stabilized against degradation. Typically, the siRNA will be in the form of isolated siRNA comprising short double-stranded RNA from about 17 nucleotides to about 29 nucleotides in length—such as approximately 19-25 contiguous nucleotides in length—that are targeted to a target mRNA. The siRNA comprise a sense RNA strand and a complementary antisense RNA strand annealed together by standard Watson-Crick base-pairing interactions. The sense strand comprises a nucleic acid sequence which is identical to a target sequence contained within the target mRNA. Although siRNA silencing is highly effective by selecting a single target in the mRNA, it may be desirable to design and employ two independent siRNA duplexes to control the specificity of the silencing effect.

The agent of the present invention may be capable of displaying other therapeutic properties.

The agent may be a known drug.

The agent may be or may be derived from a viral drug.

The agent may be or may be derived from an aminoglycoside—such as G418 sulphate or gentamycin.

The agent may modulate the integrity of a viral RNA transcript and/or viral RNA metabolism.

If combinations of agents are tested, then they may be tested simultaneously, separately or sequentially.

Applications/Uses

Effect of Genome Sequence Variation

The effect of sequence variation on transcript integrity can be determined (eg. in single mammalian cells). This assay will provide, for example, an instant insight whether the mutation activates aberrant splicing or trigger the nonsense-mediated decay (NMD) pathway.

Alternative Splicing

The assay can be used to investigate the molecular mechanism of alternative splicing more specifically those associated with human disease such as spinal muscular atrophy (SMA), muscular dystrophy and breast cancer and will aid to identify factors/drugs to restore normal pattern.

Inhibition of Splicing

To identify the inhibitors of viral RNA splicing including HIV, which can be used as anti-viral drugs.

Translation Termination

To investigate how human mutation affects the termination process. This assay will be useful to identify factors that promote translation read-through. Such factors can be used as drugs to treat nonsense-associated human diseases and as inducers for exogenous control of gene expression both in mammalian cells and in vivo.

NMD

To identify agent that inhibit NMD process. This assay can also be used to identify agents to protect nonsense-associated transcripts. Such transcripts immune to NMD can be used as a template to identify drugs for nonsense-associated human diseases capable of promoting the production of full-length active protein.

Regulation of Gene Expression

This system is useful to investigate the molecular mechanisms of splicing, translation termination and NMD processes and can control gene expression via modulation of these processes.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Ir1 Press; and, D. M. J. Lilley and J. E. Dahlberg, 1992, Methods of Enzymology: DNA Structure Part A: Synthesis and Physical Analysis of DNA Methods in Enzymology, Academic Press.

Further Aspects

There is also described a method for identifying at least one agent that modulates splicing comprising the use of a nucleic acid construct comprising at least two different reporters separated by an in frame splicing unit, wherein said splicing unit comprises at least two exons separated by at least one intron, wherein a difference in reporter activity in a cell comprising the nucleic acid construct that has been exposed to the agent in comparison to a cell that has not been exposed to the agent is indicative that said agent modulates splicing.

There is also described a method for identifying at least one agent that modulates splicing comprising the use of a nucleic acid construct comprising at least two different reporters separated by an in frame splicing unit, wherein said splicing unit comprises at least two exons separated by at least one intron and wherein the at least one intron comprises one or more stop codon(s) (eg. two or more or three or more), wherein a difference in reporter activity in a cell comprising the nucleic acid construct that has been exposed to the agent in comparison to a cell that has not been exposed to the agent is indicative that said agent modulates splicing.

The invention will now be further described by way of Examples, which are meant to serve to assist one of ordinary skill in the art in carrying out the invention and are not intended in any way to limit the scope of the invention.

### Examples

### Example 1

### Materials and Methods

Single-cell based dual-fluorescence splicing assay constructs were generated by replacing exon 9 of plasmid pTN136 with exon 12 of BMPR2 gene using PCR mutagenesis (14). The resultant splicing unit was cloned into Xho I and Bam HI sites of DsRed Express-GFP vector (Siskoglu and Nasim, unpublished data) and designated as pTN139. Further plasmids pTN140, pTN141, pTN142 and pTN143 bearing mutations in exon 12 at position c.2292insA, c.2386delG, c.2620G>T and c.2695C>T, respectively were generated. A double intron splicing unit was constructed by fusing an additional splicing unit of HC9 (15) at the 3′end of the Ad-Exon 12 unit of pTN136. The splicing unit of HC9 comprises the last 17 nucleotides of the non-muscle exon (NM) of human TPM3 gene together with the following 80-nucleotide intron, which was joined to a fragment of human dystrophin gene containing the last 260 nucleotides of intron 11 followed by the 100 nucleotides of exon 12. The double-intron splicing unit was cloned into EcoRI and KpnI sites of DsRed-Express-GFP vector and designated as pTN153. Plasmid pTN155 is a derivative of pTN153 and harbours a mutation in exon 12 at position c.2386delG.

Constructs for determining NMD were generated by fusing the double-intron splicing unit of pTN155 at the end of β-galactosidase reporter gene. The double-intron splicing intron was cloned in two steps into pBPLUGA (16). First the splicing unit of HC9 as described above was PCR amplified and cloned into KpnI and BamHI sites of pBPLUGA. The splicing unit of pTN136 was then cloned into SalI/XhoI and BamHI sites of the newly constructed plasmid and designated as pTN148. Plasmid pTN147 contains a mutation at c.2292insA.

Gene Transfer, cell culture, enzymatic assay, fluorescence microscopy and fluorescence activated cell sorting (FACS) were carried as described elsewhere (11), (15), (17). Amount of plasmid DNA transfected into HEK293 cell line varied from (from 30 ng to 1 μg) assay to assay.

### Example 2

### Results

A single-cell based dual-fluorescence assay system for the functional characterization of PAH causing BMPR2 sequence variation

GENSCAN (18) (http://genes.mit.edu/GENSCAN.html), an algorithm simulates gene prediction based on multiple parameters, including nucleotide composition, transcription, splicing and translation signals. The program predicts that the insertion (c.2292insA), deletion (c.2386delG) and nonsense mutations (c.2695C>T) into the exon 12 of BMPR2 each may alter the strength of 5′ and 3′splice sites, exon score probability, T-score and the length of the exon (FIG. 1A) probably by activating cryptic splice sites (FIG. 1B). If correct this would lead to the skipping of the nonsense codon and hence maintain a reading frame (FIG. 1C). So as to investigate the consequence of these mutations we developed a dual-fluorescence-based assay system. Exon 12 is introduced into the construct in a manner such that successful splicing would lead to the production of DsRed-Express-GFP fusion protein (FIG. 2B). Expression of the upstream reporter would only occur should a nonsense codon within exon 12 be recognized as a translation stop signal. However, should the nonsense trigger aberrant splicing, a shorter form of the fusion protein would be produced. Data from fluorescence microscopy revealed that the wild type sequence underwent splicing leading to the expression of both fluorescence proteins (FIG. 2C). However, each of the BMPR2 mutations tested namely c.2292insA, c.2386delG, and c.2695C>T failed to produce the dual-fluorescence reporter indicating that mutation had led to the incorporation of a translation stop signal within exon 12. Direct RNA analysis by means of reverse transcriptase PCR (RT-PCR) confirmed that the dual-fluorescence was consequent upon efficient splicing of wild type sequence (FIG. 2C). Western blot analysis detected a band corresponding to the size of the fusion protein in wild type construct (FIG. 2D). In contrast, the mutated construct (139-PTC; pE874X) failed to produce a band corresponding to the fusion protein confirming that the nonsense does not trigger aberrant splicing but introduced a PTC into the BMPR2 gene.

Splicing in most mammalian genes involves a process known as exon definition (19), in which an exon surrounded by introns is seen as the basic unit recognized by splicing factors. A potential limitation of the dual-fluorescence assay as described is that the nonsense mutation may not stimulate aberrant splicing due to the presence of single intron with failure to stimulate full-length exon skipping event. We therefore developed a dual-intron based reporter where an additional splicing unit from HC 9 (15) was introduced (FIG. 3A and see materials and methods). As a consequence and in the event of exon 12 incorporation or skipping, dual fluorescence could be generated whilst the presence of the nonsense, if recognized as a translational termination signal would enable DsRed expression alone. Fluorescence microscopy indicated that the wild type sequence undergoes a splicing event leading to dual fluorescence (FIG. 3B). In contrast, the deletion mutant (c.2386delG) failed to produce dual-fluorescence suggesting that the PTC did not stimulate aberrant splicing, but rather was recognized as translation stop signal. RT-PCR analysis confirmed that dual-fluorescence was derived as a consequence of exon incorporation and the mutant c.2386delG did not trigger an exon skipping event (FIG. 3C).

### Example 3

### Mutations Containing PTCs are Subject to Nonsense-Mediated RNA Decay

As the data indicated that the presence of the nonsense mutation did not stimulate aberrant splicing, we next wished to investigate whether nonsense bearing mutations of BMPR2 triggered NMD. To study this, double intron splicing unit containing either c.2386delG or c.2292insA was introduced at the 3′end of the β-galactosidase gene in such a way that the incorporation of the PTC would lead to the truncation of a full-length protein (FIG. 4A). Thus, if the mRNA is subjected to NMD, inhibition of translation using cycloheximide or puromycin would lead to the increased level of PTC bearing mRNA and therefore the level of the truncated protein would also be increased. Following transfection significant increase in the reporter protein was observed after treating with cycloheximide (5 fold) and puromycin (20 fold) (FIG. 4B). Further confirmation that these mutations were subject to NMD, plasmids encoding siRNA that inhibit expression of hUpf1 (9) were transfected together with the reporter plasmid. Compared with cells transfected with the reporter alone, the gal-luc ratio increased between 3 and 6 fold when hUpf1 RNA was knock down by RNAi (FIG. 4C).

### Example 4

### Promotion of Translation Readthrough

We demonstrated that nonsense mutation introduced pre-mature termination codon into BMPR-II protein producing insufficient level of protein providing unequivocal evidence that haplo-insufficiency is the major molecular consequence of PAH mutations. In addition to PAH, there are also more than 1800 genetic disorders caused by nonsense mutation. Two approaches are currently available to overcome nonsense-associated genetic diseases. The first approach is gene therapy, although possesses a bright future, it is still far from achieving clinical success. The aim of other approach is to suppress pre-mature termination; in other words to promote translation readthrough. As the dual-fluorescence assay was successfully utilized to characterize the PTC, we next wished to investigate whether the system can be used to identify factors that promote translation readthrough. To achieve this, we introduced a nonsense mutation into the exon 12 of the BMPR2 gene in such a way that it incorporates a PTC (FIG. 5A). In the event of readthrough, a fusion dual-fluorescence protein (DsRed Express and GFP) will be produced, whilst translation termination generates only DsRed-Express protein. Thus the ratio of two fluorescence intensities determines the efficiency of readthrough. This construct generated DsRed-Express protein following transfection into 293 cells (FIG. 5B). As aminoglycosides have been shown to promote translation readthrough, we treated the cells with G418 antibiotic. Surprisingly, 5 days after treatment cells generated dual-fluorescence. To determine the efficiency in single cells, reporter constructs were transfected and cells expressing fluorescence proteins were sorted using a fluorescence activated cell sorter (FACS). The data from FACS indicated that the construct pTN139 generated the highest level of dual-fluorescence cells. In contrast, pTN142 construct generated little or no dual-fluorescence cells (FIG. 5C). Interestingly, following gentamicin and G418 treatment, significantly high population of dual-fluorescence cells were observed (FIGS. 5Cvi and F). To determine the ratio of DsRed-Express and GFP production, which represents the efficiency of readthrough, we used a fluorescence monochrome and filter plate readers. Consistently, the data from plate readers confirmed that both gentamicin and G418 sulphate promoted translation readthrough (data not shown).

To further validate the efficiency determined by fluorescence assays, we employed an enzyme-based reporter assay. The efficiency of these chemicals has been determined before using reporters typically rely on single reporter functions and are susceptible to variations between samples particularly in regard to levels of transcription, processing, RNA stability and translation. To overcome these difficulties we employed a dual-reporter based on genes encoding luciferase and beta-galactosidease proteins, which minimized the variation confounding single reporter functions. The reporter contains an in-frame stop signal in between these two genes. In the event of translation termination, β-gal protein is generated, whilst readthrough produces a β-gal and luciferase fusion protein. The ratio of these two proteins thus determines the efficiency of readthrough (FIG. 5D). Using two different types of constructs, we confirmed that both gentamicin and G418 sulphate promoted readthrough efficiency (FIG. 5E).

A potential complication of the fluorescence approach was that of an autofluorescence which might interfere with the GFP fluorescence. To address this potential limitation, another set of vector was constructed. In this set of vectors the GFP and DsRed-Express reporters were swapped in such that the GFP is expressed regardless of translation readthrough, whilst the readthrough activates the expression of DsRed-Express protein (FIG. 7A). As expected. following transfection into HEK293 cells the PTC harbouring construct generated the GFP protein (FIG. 7B). The reporter produced both GFP and DsRed-Express dual-fluorescences once the PTC codon was changed to a sense codon.

We next wished to determine the ratio of DsRed-Express and GFP production, which represents the efficiency of readthrough (FIG. 8A). Cells were grown into a black plate. 24 hours after seeding cells were transfected and 24 hours after transfection cells were treated with various chemicals in a medium containing 0.1% fetal bovine serum. The cells were further grown for an additional 3-5 days. The growth medium was discarded, cells were washed with PBS and the fluorescence intensities were determined using a fluorescence (BMG Labtech) plate reader. Consistent with the results derived from FACS analyses, the data from plate reader confirmed that gentamicin promoted translation readthrough (FIG. 8B).

Discussion

The definitive test of whether a genome sequence variation affects transcript integrity requires RNA analysis of affected tissues which are often not available. Alternatively, the effect of mutation can be characterized using transient transfection of minigenes (15) in mammalian cells or in vitro splicing assays, comparing the splicing patterns of mutant and wild type exons. Majority of genome sequence variations include nonsense, insertion, deletion and substitution, each of them is predicted to incorporate pre-mature termination codon (PTC) in the transcript. The PTC bearing transcripts may be degraded by nonsense-mediated decay. PTCs have also been reported to inhibit splicing and cause altered splice site selection in pre-mRNAs (20), (21), (22), (23). Functional characterizations of sequence variation thus rely on the understanding whether the variation activates aberrant splicing or triggers NMD pathway.

We previously developed a double reporter splicing assay system (11) which is suitable for determining splicing efficiency but is not suitable for investigating the effect of mutation on alternative splicing. To identify cis or trans-acting factors that affect alternative splicing we developed a minigene system (15) which required direct RNA analysis and hence a large number of samples can not be handled at a time. To overcome these difficulties we developed a novel fluorescence activity based assay systems (single and double introns) aiming to characterize the factors that influence RNA metabolism. The single cell based assay system is based on red (DsRed-Express) and green (GFP) fluorescence proteins. DsRed-Express and GFP were chosen as their excitation peaks are 557 and 488, respectively and therefore can easily be distinguished under a fluorescence microscope and their level of expression can be determined by means of flow cytometry (17). As each of these proteins is stable, exhibiting no detectable photoinstability with a low signal-to-noise ratio (24) and a shorter maturation time than that of other fluorescence proteins including DsRed, the single cell based assays could be useful to investigate the integrity and efficiency of splicing in the early stages of developing embryos. The dual-fluorescence assay is directly measured on intact cell and does not require cell wall disruption or addition of a substrate and the sensitivity is comparable to that with gal-luc assay and therefore is suitable for high-throughput screening of small molecules, peptides and nucleic acids that might affect a specific splicing interaction. Another potential advantage over the existing assays is that these assays are based on double reporter principle and hence bypass the variables confounding single reporter (11), (12). Recently a bichromatic reporter based on the alternative reading frames of fluorescence proteins has been developed (25). In this system, the exon incorporation generated a green fluorescence protein (GFP) whilst exon skipping produced a red fluorescence protein (dsRed) providing a dual-fluorescence equivalent of single reporter.

Using the dual-fluorescence assay we were able to show that mutations at position c.2292insA, c.2386delG, and c.2695C>T did not activate aberrant splicing as predicted by computer algorithm (FIGS. 2 and 3). Since the nonsense mutations failed to stimulate aberrant splicing, we next investigated if the PTC triggered NMD. We developed a NMD assay system based on β-galactosidase reporter gene. We tested the NMD assay in several ways to show that it reflects the efficiency of NMD. The highest levels of reporter activity were observed when cells were treated with common NMD inhibitors including cycloheximide or puromycin (FIG. 4A). The reporter activities also increased when cells were transfected with siRNAs that knock down an essential gene Upf1 required for NMD (FIG. 4B). By quantitative RT-PCR using the mouse and human smooth muscle cell lines harbouring nonsense mutations (26), (27) we confirmed that nonsense mutations do trigger the NMD pathway (data not shown).

Appreciation of the mechanisms that influence RNA metabolism may promote the development of therapeutic strategies that can be applied to broad classes of human diseases. The majority of PTC bearing mutations identified in human diseases follow the NMD pathway and as a result produces insufficient levels of full-length protein. Intriguing attempts have been made to treat genetic disorders resulting from PTCs with aminoglycosides and small molecule (PTC124) aimed at promoting nonsense codon readthrough (28), (29). We and others developed enzymatic activity based reporters (13), (30) to determine the efficiency of translation readthrough. The readthrough efficiencies achieved by aminoglycosides and PTC124 are as low as 2-10%, offers opportunities for identification for more efficient factors. The major drawback for the identification of such factors using these systems is that the assay can not be measured directly on intact cells and requires cell wall disruption or addition of a substrate and hence offers a limited opportunity for high-throughput screening. In addition, chemicals such as PTC124 that modulate the enzymatic activities might generate biased readouts (31). To identify factors that promote translation readthrough, our dual-fluorescence assay (see FIG. 5) has advantages over the enzyme based reporters as (i) the assay does not require cell lysis, (ii) addition of a substrate to measure reporter activities, (iii) the readouts are not dependent on enzymatic acitivities and hence the chemical-reporter enzyme interactions are minimal and finally, (iv) the assay is measured directly in intact living cells. As the dual-fluorescence system is suitable for high-throughput screening of small molecules, peptide and nucleic acids, it may be useful in isolation of factors that achieve the maximum readthrough efficiency. Such molecules with improved suppression activity and novel pharmacogenetic properties should expand the utility of inducers to control translation-based gene regulation strategies (32) and may have important clinical applications in the setting of gene therapy. However, a potential complication of the GFP based fluorescence approach was that of an autofluorescence which might interfere with the GFP fluorescence. This is addressed by swapping the GFP and DsRed-Express reporters, which has no or little background autofluorescence.

Our single cell based assay (FIGS. 2, 3 and 6A) may prove to be useful to study a wide range of alternatively spliced genes linked to human diseases such as spinal muscular atrophy (SMA). SMA is caused by homogygous loss of survival motor neuron gene (SMN1) (33). SMN2, a nearly identical copy of SMN1, is preserved in SMA patients. A single nucleotide difference between these two genes causes exon 7 skipping of SMN2, produces a non-functional protein and thus can not compensate the loss of SMN1. Drugs or transacting factors that could increase the incorporation of exon 7 of SMN2 would provide protection from the disease. Since the dual-fluorescence splicing assay is suitable for high-throughput screening for cDNA, peptide and chemical libraries, it will be useful to identify trans-acting factors that sufficiently incorporate exon 7 of the SMN2. In addition, the system could be widely used to isolate drugs for number of other human diseases that affect specific splicing reaction (34), (35).

Extensive genetic variations have been found within the genome sequence of some pathogenic viruses such as HIV which allow the emergence of drug-resistant viruses. As a result, the efficacy of anti-viral drugs is compromised. An effective option for developing anti-viral therapy is to target viral replication. Viral RNA splicing is a crucial step of viral life cycle and allows the production of some key proteins. Recently, a therapeutic agent for HIV-1 has been developed based on inhibition of HIV-1 splicing. Our assays (FIGS. 2, 3 and 6B) offer an excellent opportunity to isolate more effective splicing inhibitors which can be used as anti-viral drugs. In contrast, agents that modulate alternative splicing of viral RNA can be used to engineer replication-competent viruses to treat cancer.

The current system (FIGS. 3ii 4 and 6DE) also offers the possibility for innovative and useful treatments based on approaches specifically to inhibit disease associated NMD transcripts. An advantage of the NMD fluorescence (see FIG. 3ii) is that the system based on DsRed-Express by comparison to GFP-based approach (9) encounters much reduced autofluorescence. The chemiluminescence based NMD assay is sensitive (see FIG. 4) and thus can quantify the efficiency of NMD. Recently, a similar approach based on luciferase activity has been reported to monitor NMD in Hela cells (10). However, the single cell based system is suitable for high-throughput screening of small molecule and RNAi libraries, whilst the luminescence based assay provide a platform for validating the potential hits making this system very useful to identify and investigate the trans-acting factor and signaling pathways affecting NMD.

### REFERENCES

1. Kuzmiak, H. A. and Maquat, L. E. (2006) Applying nonsense-mediated mRNA decay research to the clinic: progress and challenges. Trends Mol Med, 12, 306-316.

2. Frischmeyer, P. A. and Dietz, H. C. (1999) Nonsense-mediated mRNA decay in health and disease. Hum Mol Genet, 8, 1893-1900.

3. Maquat, L. E. (2004) Nonsense-mediated mRNA decay: splicing, translation and mRNP dynamics. Nat Rev Mol Cell Biol, 5, 89-99.

4. Wang, J., Chang, Y. F., Hamilton, J. I. and Wilkinson, M. F. (2002) Nonsense-associated altered splicing: a frame-dependent response distinct from nonsense-mediated decay. Mol Cell, 10, 951-957.

5. Mendell, J. T. and Dietz, H. C. (2001) When the message goes awry: disease-producing mutations that influence mRNA content and performance. Cell, 107, 411-414.

6. Disset, A., Bourgeois, C. F., Benmalek, N., Claustres, M., Stevenin, J. and Tuffery-Giraud, S. (2006) An exon skipping-associated nonsense mutation in the dystrophin gene uncovers a complex interplay between multiple antagonistic splicing elements. Hum Mol Genet, 15, 999-1013.

7. Zhang, M. L., Lorson, C. L., Androphy, E. J. and Zhou, J. (2001) An in vivo reporter system for measuring increased inclusion of exon 7 in SMN2 mRNA: potential therapy of SMA. Gene Ther, 8, 1532-1538.

8. Abes, S., Williams, D., Prevot, P., Thierry, A., Gait, M. J. and Lebleu, B. (2006) Endosome trapping limits the efficiency of splicing correction by PNA-oligolysine conjugates. J Control Release, 110, 595-604.

9. Paillusson, A., Hirschi, N., Vallan, C., Azzalin, C. M. and Muhlemann, O. (2005) A GFP-based reporter system to monitor nonsense-mediated mRNA decay. Nucleic Acids Res, 33, e54.

10. Boelz, S., Neu-Yilik, G., Gehring, N. H., Hentze, M. W. and Kulozik, A. E. (2006) A chemiluminescence-based reporter system to monitor nonsense-mediated mRNA decay. Biochem Biophys Res Commun, 349, 186-191.

11. Nasim, M. T., Chowdhury, H. M. and Eperon, I. C. (2002) A double reporter assay for detecting changes in the ratio of spliced and unspliced mRNA in mammalian cells. Nucleic Acids Res, 30, e109.

12. Nasim, M. T. and Eperon, I. C. (2006) A double-reporter splicing assay for determining splicing efficiency in mammalian cells. Nat Protoc, 1, 1022-1028.

13. Nasim, M. T., Jaenecke, S., Belduz, A., Kollmus, H., Flohe, L. and McCarthy, J. E. (2000) Eukaryotic selenocysteine incorporation follows a nonprocessive mechanism that competes with translational termination. J Biol Chem, 275, 14846-14852.

14. Nasim, M. T., Eperon, I. C., Wilkins, B. M. and Brammar, W. J. (2004) The activity of a single-stranded promoter of plasmid ColIb-P9 depends on its secondary structure. Mol Microbiol, 53, 405-417.

15. Nasim, M. T., Chemova, T. K., Chowdhury, H. M., Yue, B. G. and Eperon, I. C. (2003) HnRNP G and Tra2beta: opposite effects on splicing matched by antagonism in RNA binding. Hum Mol Genet, 12, 1337-1348.

16. Kollmus, H., Flohe, L. and McCarthy, J. E. (1996) Analysis of eukaryotic mRNA structures directing cotranslational incorporation of selenocysteine. Nucleic Acids Res, 24, 1195-1201.

17. Nasim, M. T. and Trembath, R. C. (2005) A dual-light reporter system to determine the efficiency of protein-protein interactions in mammalian cells. Nucleic Acids Res, 33, e66.

18. Burge, C. and Karlin, S. (1997) Prediction of complete gene structures in human genomic DNA. J Mol Biol, 268, 78-94.

19. Robberson, B. L., Cote, G. J. and Berget, S. M. (1990) Exon definition may facilitate splice site selection in RNAs with multiple exons. Mol Cell Biol, 10, 84-94.

20. Carter, M. S., Li, S. and Wilkinson, M. F. (1996) A splicing-dependent regulatory mechanism that detects translation signals. Embo J, 15, 5965-5975.

21. Gersappe, A., Burger, L. and Pintel, D. J. (1999) A premature termination codon in either exon of minute virus of mice P4 promoter-generated pre-mRNA can inhibit nuclear splicing of the intervening intron in an open reading frame-dependent manner. J Biol Chem, 274, 22452-22458.

22. Gersappe, A. and Pintel, D. J. (1999) A premature termination codon interferes with the nuclear function of an exon splicing enhancer in an open reading frame-dependent manner. Mol Cell Biol, 19, 1640-1650.

23. Brogna, S. (2001) Pre-mRNA processing: insights from nonsense. Curr Biol, 11, R838-841.

24. Finley, K. R., Davidson, A. E. and Ekker, S. C. (2001) Three-color imaging using fluorescent proteins in living zebrafish embryos. Biotechniques, 31, 66-70, 72.

25. Orengo, J. P., Bundman, D. and Cooper, T. A. (2006) A bichromatic fluorescent reporter for cell-based screens of alternative splicing. Nucleic Acids Res, 34, e148.

26. Beppu, H., Kawabata, M., Hamamoto, T., Chytil, A., Minowa, O., Noda, T. and Miyazono, K. (2000) BMP type II receptor is required for gastrulation and early development of mouse embryos. Dev Biol, 221, 249-258.

27. Yang, X., Lee, P. J., Long, L., Trembath, R. C. and Morrell, N. W. (2007) BMP4 Induces HO-1 via a Smad Independent, p38MAPK Dependent Pathway in Pulmonary Artery Myocytes. Am J Respir Cell Mol Biol.

28. Linde, L., Boelz, S., Nissim-Rafinia, M., Oren, Y. S., Wilschanski, M., Yaacov, Y., Virgilis, D., Neu-Yilik, G., Kulozik, A. E., Kerem, E. et al. (2007) Nonsense-mediated mRNA decay affects nonsense transcript levels and governs response of cystic fibrosis patients to gentamicin. J Clin Invest, 117, 683-692.

29. Welch, E. M., Barton, E. R., Zhuo, J., Tomizawa, Y., Friesen, W. J., Trifillis, P., Paushkin, S., Patel, M., Trotta, C. R., Hwang, S. et al. (2007) PTC124 targets genetic disorders caused by nonsense mutations. Nature, 447, 87-91.

30. Bidou, L., Hatin, I., Perez, N., Allamand, V., Panthier, J. J. and Rousset, J. P. (2004) Premature stop codons involved in muscular dystrophies show a broad spectrum of readthrough efficiencies in response to gentamicin treatment. Gene Ther, 11, 619-627.

31. Auld, D. S., Thome, N., Maguire, W. F. and Inglese, J. (2009) Mechanism of PTC124 activity in cell-based luciferase assays of nonsense codon suppression. Proc Natl Acad Sci USA.

32. Murphy, G. J., Mostoslavsky, G., Kotton, D. N. and Mulligan, R. C. (2006) Exogenous control of mammalian gene expression via modulation of translational termination. Nat Med, 12, 1093-1099.

33. Cartegni, L. and Krainer, A. R. (2002) Disruption of an SF2/ASF-dependent exonic splicing enhancer in SMN2 causes spinal muscular atrophy in the absence of SMN1. Nat Genet, 30, 377-384.

34. Cartegni, L., Chew, S. L. and Krainer, A. R. (2002) Listening to silence and understanding nonsense: exonic mutations that affect splicing. Nat Rev Genet, 3, 285-298.

35. Wang, G. S. and Cooper, T. A. (2007) Splicing in disease: disruption of the splicing code and the decoding machinery. Nat Rev Genet.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for determining the effect of a genetic variation on the integrity of an RNA transcript comprising the use of a nucleic acid construct comprising two different reporters separated by an in frame splicing unit, wherein said splicing unit comprises at least two exons separated by at least one intron and wherein at least one exon comprises a genetic variation, wherein a difference in reporter activity in a cell comprising the nucleic acid construct in comparison to a cell comprising a nucleic acid construct without the genetic variation is indicative that said genetic variation affects the integrity of the RNA transcript.

2. The method according to claim 1, comprising the steps of:
(a) providing a nucleic acid construct comprising two different reporters separated by an in frame splicing unit, wherein said splicing unit comprises at least two exons separated by at least one intron and wherein at least one exon comprises the genetic variation;
(b) transfecting said construct into a cell and/or generating a stable cell line;
(c) culturing said cell; and
(d) determining the effect of said genetic variation on the integrity of the RNA transcript,
wherein a difference in reporter activity in comparison to a cell comprising the nucleic acid construct without the genetic variation is indicative that said genetic variation affects the integrity of the RNA transcript.

3. The method according to claim 1 or claim 2, wherein expression of both reporters is indicative that said genetic variation does not terminate transcription and is not a premature termination codon.

4. The method according to claim 1 or claim 2, wherein expression of the single reporter upstream of the splicing unit is indicative that said genetic variation terminates transcription and is a premature termination codon.

5. The method according to claim 1 or claim 2, wherein step (a) comprises providing a nucleic acid construct comprising two different reporters separated by an in frame splicing unit, wherein said splicing unit comprises three exons separated by two introns, wherein at least one exon (eg. the exon separated by the two introns) comprises the genetic variation.

6. The method according to claim 5, wherein expression of both reporters is indicative that said genetic variation results in exon incorporation or exon skipping and is not a premature termination codon.

7. The method according to claim 5 or claim 6, wherein exon incorporation and/or exon skipping are further distinguished using RT-PCR.

8. The method according to claim 5, wherein expression of the single reporter upstream of the splicing unit in combination with the exons upstream of said genetic variation is indicative that said genetic variation is a premature termination codon and that the RNA transcript is subject to nonsense mediated decay.

9. The method according to any of the preceding claims, wherein the genetic variation is a mutation or single nucleotide polymorphism (SNP).

10. The method according to claim 9, wherein the mutation is a nonsense mutation, an insertion mutation, a deletion mutation, a duplication or a substitution mutation.

11. The method according to any of the preceding claims, wherein the reporters are fluorescent reporters.

12. The method according to claim 11, wherein at least one of the reporters is DsRedExpress.

13. The method according to claim 11 or claim 12, wherein at least one of the reporters is GFP.

14. A method for identifying at least one agent that modulates splicing comprising the use of a nucleic acid construct comprising at least two different reporters separated by an in frame splicing unit, wherein said splicing unit comprises at least two exons separated by at least one intron and wherein the at least one intron does not comprise at least one stop codon, wherein a difference in reporter activity in a cell comprising the nucleic acid construct that has been exposed to the agent in comparison to a cell that has not been exposed to the agent is indicative that said agent modulates splicing.

15. The method according to claim 14, comprising the steps of:
(a) providing a nucleic acid construct comprising at least two different reporters separated by an in frame splicing unit, wherein said splicing unit comprises at least two exons separated by at least one intron and wherein the at least one intron does not comprise one or more stop codon(s);
(b) transfecting said construct into a cell and/or generating stable cell line;
(c) culturing said cell;
(d) contacting said cell with at the least one agent; and
(e) determining the effect of said agent on the splicing of RNA;
wherein a difference in reporter activity in comparison to a cell that has not been exposed to the at least one agent is indicative that said agent modulates splicing.

16. The method according to claim 14 or claim 15, wherein expression of both reporters in the cell exposed to the agent and the expression of a single reporter in the cell not exposed to the agent is indicative that said agent promotes normal splicing.

17. The method according to claim 14 or claim 15, wherein an increase in the expression of both reporters in the cell exposed to the agent in comparison to the cell not exposed to the agent is indicative that said agent promotes normal splicing.

18. The method according to claim 14 or claim 15, wherein expression of a single reporter in the cell exposed to the agent and the expression of both reporters in the cell not exposed to the agent is indicative that said agent promotes splicing inhibition.

19. The method according to claim 14 or claim 15, wherein an increase in the expression of the single reporter in the cell exposed to the agent in comparison to the cell not exposed to the agent is indicative that said agent promotes splicing inhibition.

20. A method for identifying at least one agent that modulates translation comprising the use of a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon, wherein a difference in reporter activity in a cell comprising the nucleic acid construct that is exposed to the agent in comparison to a cell that has not been exposed to the agent is indicative that said agent modulates translation.

21. The method according to claim 20, comprising the steps of:
(a) providing a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon;
(b) transfecting said construct into a cell and/or establishing a stable cell line;
(c) culturing said cell;
(d) contacting said cell with the at least one agent; and
(e) determining the effect of said agent on the splicing of RNA;
wherein a difference in reporter activity in comparison to a cell that has not been exposed to the at least one agent is indicative that said agent modulates translation.

22. The method according to claim 20 or claim 21, wherein expression of both reporters in the cell exposed to the agent and the expression of a single reporter in the cell not exposed to the agent is indicative that said agent promotes translational read-through.

23. The method according to claim 20 or claim 21, wherein an increase in the expression of both reporters in the cell exposed to the agent in comparison to the cell not exposed to the agent is indicative that said agent promotes translation read-through.

24. The method according to claim 20 or claim 21, wherein expression of a single reporter in the cell exposed to the agent and the expression of both reporters in the cell not exposed to the agent is indicative that said agent promotes translation termination.

25. The method according to claim 20 or claim 21, wherein an increase in the expression of the single reporter in the cell exposed to the agent in comparison to the cell not exposed to the agent is indicative that said agent promotes translation termination.

26. A method for identifying at least one agent that modulates translation and/or nonsense mediated decay comprising the use of a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon, wherein a difference in reporter activity in a cell comprising the nucleic acid construct that is exposed to the agent in comparison to a cell that has not been exposed to the agent is indicative that said agent modulates translation and nonsense mediated decay.

27. A method for identifying an agent that modulates translation and/or nonsense mediated decay comprising the steps of:
(a) providing a nucleic acid construct comprising at least two different reporters separated by an in frame splicing unit, wherein said splicing unit comprises at least two exons separated by at least one intron and wherein at least one exon (eg. the exon downstream of said intron) comprises a premature termination codon;
(b) transfecting said construct into a cell and/or establishing a stable cell line;
(c) culturing said cell;
(d) contacting said cell with at least one agent; and
(e) determining the effect of said agent on the splicing of RNA;
wherein a difference in reporter activity in comparison to a cell that has not been exposed to the at least one agent is indicative that said agent modulates translation and nonsense mediated decay.

28. The method according to claim 26 or claim 27, wherein expression of both reporters in the cell exposed to the agent and the expression of a single reporter in the cell not exposed to the agent is indicative that said agent promotes translational read-through and inhibits nonsense mediated decay.

29. The method according to claim 26 or claim 27, wherein an increase in the expression (ratio) of both reporters in the cell exposed to the agent in comparison to the cell not exposed to the agent is indicative that said agent promotes translational read-through and inhibits nonsense mediated decay.

30. The method according to claim 26 or claim 27, wherein expression of a single reporter in the cell exposed to the agent and the expression of both reporters in the cell not exposed to the agent is indicative that said agent promotes translation termination.

31. The method according to claim 26 or claim 27, wherein an increase in the expression of the single reporter in the cell exposed to the agent in comparison to the cell not exposed to the agent is indicative that said agent promotes translation termination.

32. A method for identifying at least one agent that modulates exon incorporation or exon skipping comprising the use of a nucleic acid construct comprising at least two different reporters separated by an in frame splicing unit, wherein said splicing unit comprises at least three exons separated by at least two introns, wherein a difference in reporter activity in a cell comprising the nucleic acid construct that is exposed to the agent in comparison to a cell that has not been exposed to the at least one agent is indicative that said agent modulates exon skipping or exon incorporation.

33. The method according to claim 32 comprising the steps of:
(a) providing a nucleic acid construct comprising at least two different reporters separated by an in frame splicing unit, wherein said splicing unit comprises at least three exons separated by at least two introns;
(b) transfecting said construct into a cell and/or establishing a stable cell line;
(c) culturing said cell;
(d) contacting said cell with at least one agent; and
(e) determining the effect of said mutation on exon skipping;
wherein a difference in reporter activity in comparison to a cell that has not been exposed to the at least one agent is indicative that said agent modulates exon skipping or exon incorporation.

34. The method according to claim 32 or claim 33, wherein expression of both reporters in the cell exposed to the agent and the expression of a single reporter in the cell not exposed to the agent is indicative that said agent promotes exon incorporation.

35. The method according to claim 32 or claim 33, wherein an increase in the expression of both reporters in the cell exposed to the agent in comparison to the cell not exposed to the agent is indicative that said agent promotes exon incorporation.

36. The method according to claim 32 or claim 33, wherein expression of a single reporter in the cell exposed to the agent and the expression of both reporters in the cell not exposed to the agent is indicative that said agent promotes exon skipping and/or alternative splice site selection.

37. The method according to claim 32 or claim 33, wherein an increase in the expression of the single reporter in the cell exposed to the agent in comparison to the cell not exposed to the agent is indicative that said agent promotes exon skipping and/or alternative splice site selection.

38. A method for identifying at least one agent that modulates exon incorporation or exon skipping or nonsense mediated decay comprising the use of a nucleic acid construct comprising two different reporters separated by an in frame splicing unit, wherein said splicing unit comprises three exons separated by two introns, wherein at least one exon (eg. the exon separated by the two introns) comprises a premature termination codon, wherein a difference in reporter activity in a cell comprising the nucleic acid construct that is exposed to the agent in comparison to a cell that has not been exposed to the agent is indicative that said agent modulates exon incorporation or exon skipping or nonsense mediated decay.

39. The method according to claim 38 comprising the steps of:
(a) providing a nucleic acid construct comprising two different reporters separated by an in frame splicing unit, wherein said splicing unit comprises three exons separated by two introns, wherein at least one exon (eg. the exon separated by the two introns) comprises a premature termination codon;
(b) transfecting said construct into a cell and/or establishing a stable cell line;
(c) culturing said cell;
(d) contacting said cell with at least one agent; and
(e) determining the effect of said mutation;
wherein a difference in reporter activity in comparison to a cell that has not been exposed to the at least one agent is indicative that said agent modulates exon incorporation or exon skipping or nonsense mediated decay

40. The method according to claim 38 or claim 39, wherein expression of both reporters in the cell exposed to the agent and the expression of a single reporter in the cell not exposed to the agent is indicative that said agent promotes exon incorporation or exon skipping.

41. The method according to claim 38 or claim 39, wherein an increase in the expression of both reporters in the cell exposed to the agent in comparison to the cell not exposed to the agent is indicative that said agent promotes exon incorporation or exon skipping.

42. The method according to claim 40 or claim 41, wherein exon incorporation or exon skipping is verified by analysing the RNA transcript.

43. The method according to claim 42, wherein the RNA transcript is analyzed using RT-PCR.

44. The method according to claim 38 or claim 39, wherein expression of a single reporter in the cell exposed to the agent and the expression of both reporters in the cell not exposed to the agent is indicative that said agent promotes nonsense mediated decay.

45. The method according to claim 38 or claim 39, wherein an increase in the expression of the single reporter in the cell exposed to the agent in comparison to the cell not exposed to the agent is indicative that said agent promotes nonsense mediated decay.

46. A method for identifying at least one agent that modulates nonsense mediated decay comprising the use of a nucleic acid construct comprising two different reporters separated by an in frame splicing unit, wherein said splicing unit comprises three exons separated by two introns, wherein at least one exon (eg. the exon separated by the two introns) comprises a premature termination codon, wherein a difference in reporter activity in a cell comprising the nucleic acid construct that has been exposed to the agent in comparison to a cell that has not been exposed to the at least one agent is indicative that said agent modulates nonsense mediated decay.

47. The method according to claim 46, comprising the steps of:
(a) providing a nucleic acid construct comprising two different reporters separated by an in frame splicing unit, wherein said splicing unit comprises three exons separated by two introns, wherein at least one exon (eg. the exon separated by the two introns) comprises a premature termination codon;
(b) transfecting said construct into a cell and/or establishing a stable cell line;
(c) culturing said cell;
(d) contacting said cell with at least one agent; and
(e) determining the effect of said agent on nonsense mediated decay;
wherein a difference in reporter activity in comparison to a cell that has not been exposed to the at least one agent is indicative that said agent modulates nonsense mediated decay.

48. The method according to claim 46 or claim 47, wherein a low level of the reporter upstream of the splicing unit in the cell exposed to the at least one agent in comparison to the cell that has not been exposed to the at least one agent is indicative that said agent promotes nonsense mediated decay.

49. The method according to claim 46 or claim 47, wherein a high level of the reporter upstream of the splicing unit in the cell exposed to the at least one agent in comparison to the cell that has not been exposed to the at least one agent is indicative that said agent inhibits nonsense mediated decay.

50. An agent obtained or obtainable by the method according to any of claims 14 to 49.

51. A nucleic acid construct comprising two different reporters separated by an in frame splicing unit, wherein said splicing unit comprises at least two exons separated by at least one intron, with the proviso that said intron does not comprise a translation stop signal.

52. A nucleic acid construct comprising two different reporters separated by an in frame splicing unit, wherein said splicing unit comprises at least two exons separated by at least one intron and wherein at least one exon (eg. the exon downstream of said intron) comprises a mutation.

53. A nucleic acid construct comprising two different reporters separated by an in frame splicing unit, wherein said splicing unit comprises three exons separated by two introns, wherein at least one exon (eg. the exon separated by the two introns) comprises at least one mutation that causes nonsense mediated decay.

54. A vector comprising the nucleic acid construct according to any of claims 51 to 53.

55. A host cell comprising the nucleic acid construct according to any of claims 51 to 53 or the vector according to claim 54.

56. A method for determining the effect of at least one mutation on the integrity of an RNA transcript comprising the use of the nucleic acid construct according to any of claims 51 to 53, the vector according to claim 54 or the cell according to claim 55.

57. A method for identifying an agent that modulates the integrity of an RNA transcript comprising the use of the nucleic acid construct according to any of claims 51 to 53, the vector according to claim 54 or the cell according to claim 55.

58. Use of the nucleic acid construct according to any of claims 51 to 53, the vector according to claim 54 or the cell according to claim 55 for determining the effect of at least one mutation on the integrity of an RNA transcript.

59. Use of the nucleic acid construct according to any of claims 51 to 53, the vector according to claim 54 or the cell according to claim 55 for identifying an agent that modulates the integrity of an RNA transcript.

60. Mutation c.2292insA in exon 12 of BMPR2.

61. Mutation c.2386delG in exon 12 of BMPR2.

62. Mutation c.2695C>T in exon 12 of BMPR2.

63. Mutation c.2386delG in exon 12 of BMPR2.

64. Mutation c.2620G>T in exon 12 of BMPR2.

65. A method, an agent, a nucleic acid construct, a vector, a host cell, a use or a mutation as described herein with reference to the accompanying description and drawings.

66. The method according to claim 11, wherein at least one of the reporters is DsRedExpress and at least one of the reporters is GFP and wherein DsRedExpress reporter is downstream of the GFP reporter.


SEQUENCE LISTING (16 sequences)

SEQ ID NO: 1 - DNA, 88 bp, Adenovirus type 2
gtactccctctcaaaagcgggcatgacttctgcgctaagattgtcagttccaaaaacgaggaggatttgatattcaccagctgttggg

SEQ ID NO: 2 - DNA, 136 bp, Adenovirus type 2
gtgagtcctttgagggtggccgcgtccatctggtcagaaaagacaatctttttgttgtcaagcttgctgcacgtctagggcgcagtagtccagggtttccttgatgatgtcatacttatcctgtcccttttttttc

SEQ ID NO: 3 - DNA, 99 bp, Homo sapiens
tttaacatagttttaatggatctccagaatcagaaagaaagagttgaatgactggctaacaaaaacagaagaaagaacaaggaatggaggaagagcctc

SEQ ID NO: 4 - DNA, 51 bp, Artificial Sequence
  Description of Artificial Sequence Synthetic oligonucleotide
acctcatgtggtgacagtcaccatgaatggtgtggcaggtagaaaccacag

SEQ ID NO: 5 - DNA, 24 bp, Artificial Sequence
  Description of Artificial Sequence Synthetic oligonucleotide
acctcatgtggtgacagtcaccat

SEQ ID NO: 6 - DNA, 25 bp, Artificial Sequence
  Description of Artificial Sequence Synthetic oligonucleotide
agtgacacatagggcccaagaaatg

SEQ ID NO: 7 - DNA, 21 bp, Artificial Sequence
  Description of Artificial Sequence Synthetic oligonucleotide
ccactagaaggtggccgaact

SEQ ID NO: 8 - DNA, 28 bp, Artificial Sequence
  Description of Artificial Sequence Synthetic oligonucleotide
gcaatccatgttcagaacaagatgttct

SEQ ID NO: 9 - protein, 48 aa, Homo sapiens
NSTKEPRLKFGSKHKSNLKQVETGVAKMNTINAAEPHVVTVTMNGVAG

SEQ ID NO: 10 - protein, 39 aa, Artificial Sequence
  Description of Artificial Sequence Synthetic polypeptide
NINKRAPAKIWQQAQIKLETSRNWSCQDEYNQCSRTSCG

SEQ ID NO: 11 - protein, 49 aa, Homo sapiens
NAAEPHVVTVTMNGVAGRNHSVNSHAATTQYANGTVLSGQTTNIVTHRA

SEQ ID NO: 12 - protein, 8 aa, Artificial Sequence
  Description of Artificial Sequence Synthetic peptide
NQQNLMWA

SEQ ID NO: 13 - protein, 17 aa, Homo sapiens
EGGRTNSNNNNSNPCSE

SEQ ID NO: 14 - DNA, 21 bp, Artificial Sequence
  Description of Artificial Sequence Synthetic oligonucleotide
ctcagatctcgagtactccct

SEQ ID NO: 15 - DNA, 24 bp, Artificial Sequence
  Description of Artificial Sequence Synthetic oligonucleotide
gtatacagaggatccatggcctcc

SEQ ID NO: 16 - DNA, 18 bp, Artificial Sequence
  Description of Artificial Sequence Synthetic oligonucleotide
ttcctgtagtctagataa